(19) United States
(12) Patent Application Publication
(10) Pub. No.: US 2023/0337936 A1
(43) Pub. Date: Oct. 26, 2023

(54) A method for analysing a breath sample for screening, diagnosis or monitoring of SARS-CoV-2 carriage or infection (COVID-19) on humans

(71) Applicants: UNIVERSITE DE VERSAILLES-ST QUENTIN EN YVELINES, VERSAILLES CEDEX (FR); ASSISTANCE PUBLIQUE - HOPITAUX DE PARIS, PARIS (FR); INSERM (Institut National de la Sante et de la Recherche Medicale), PARIS (FR); COMMISSARIAT A L'ENERGIE ATOMIQUE ET AUX ENERGIES ALTERNATIVES, PARIS (FR); ASSOCIATION HOPITAL FOCH, SURESNES (FR)

(72) Inventors: Camille ROQUENCOURT, PUTEAUX (FR); Etienne THEVENOT, ORSAY (FR); Djillali ANNANE, MAISON-ALFORT (FR); Stanislas GRASSIN-DELYLE, RUEIL-MALMAISON (FR)

(21) Appl. No.: 18/044,940

(22) PCT Filed: Sep. 17, 2021
(86) PCT No.: PCT/IB2021/000642

§ 371 Date: Mar. 10, 2023

(30) Foreign Application Priority Data
Sep. 17, 2020 (FR) FR2009446
Oct. 7, 2020 (EP) 20306170.0

(51) Int. Cl. A61B 5/08; A61M 16/08; A61B 5/097; G01N 33/497

(52) U.S. Cl. CPC A61B 5/082; A61M 16/085; A61B 5/097; G01N 33/497

(57) ABSTRACT

The method for analysis, the method comprising:—obtaining a sample comprising elements coming from breath exhaled from a person (112);—exposing the sample to a spectrometer;—determining at least one value of a signal intensity and/or concentration of ions defined by its mass-to-charge ratio (m/z) given by the spectrometer in at least one range, the or each range being defined by a median value and limits of the range;—applying at least one test to the value, the range(s) and the test being configured for identifying that:—a person carries and/or is infected with SARS-CoV-2,—a person does not carry and/or is not infected with SARS-CoV-2,—a person suffers from COVID-19, and/or—a person does not suffer from COVID-19; and—communicating a message according to a result of the test.

The invention relates to the SARS-CoV-2 virus carriage or infection (COVID-19) and to tests for screening, diagnosis or monitoring thereof.

The prior art includes the following documents which are referred to in this specification:

1. COVID-19 Dashboard by the Center for Systems Science and Engineering (CSSE) at Johns Hopkins University (JHU). https://coronavirus.jhu.edu/map.html (accessed Sep. 28, 2020.

2. Wiersinga W J, Rhodes A, Cheng A C, Peacock S J, Prescott H C. Pathophysiology, Transmission, Diagnosis, and Treatment of Coronavirus Disease 2019 (COVID-19): A Review. JAMA 2020.

3. Beigel J H, Tomashek K M, Dodd L E, et al. Remdesivir for the Treatment of Covid-19—Preliminary Report. N Engl J Med 2020.

4. Recovery_Collaborative_Group, Horby P, Lim W S, et al. Dexamethasone in Hospitalized Patients with Covid-19—Preliminary Report. N Engl J Med 2020.

5. Lucas C, Wong P, Klein J, et al. Longitudinal analyses reveal immunological misfiring in severe COVID-19. Nature 2020.

6. Hadjadj J, Yatim N, Barnabei L, et al. Impaired type I interferon activity and inflammatory responses in severe COVID-19 patients. Science 2020.

7. Kuri-Cervantes L, Pampena M B, Meng W, et al. Comprehensive mapping of immune perturbations associated with severe COVID-19. Sci Immunol 2020; 5(49).

8. Shen B, Yi X, Sun Y, et al. Proteomic and Metabolomic Characterization of COVID-19 Patient Sera. Cell 2020; 182 (1): 59-72 e15.

9. Kataoka H, Saito K, Kato H, Masuda K. Noninvasive analysis of volatile biomarkers in human emanations for health and early disease diagnosis. Bioanalysis 2013; 5 (11): 1443-59.

10. Rattray N J, Hamrang Z, Trivedi D K, Goodacre R, Fowler S J. Taking your breath away: metabolomics breathes life in to personalized medicine. Trends Biotechnol 2014; 32 (10): 538-48.

11. Amann A, de Lacy Costello B, Miekisch W, et al. The human volatilome: volatile organic compounds (VOCs) in exhaled breath, skin emanations, urine, feces and saliva. J Breath Res 2014; 8 (3): 034001.

12. de Lacy Costello B, Amann A, Al-Kateb H, et al. A review of the volatiles from the healthy human body. J Breath Res 2014; 8 (1): 014001.

13. Koo S, Thomas H R, Daniels S D, et al. A breath fungal secondary metabolite signature to diagnose invasive aspergillosis. Clin Infect Dis 2014; 59 (12): 1733-40.

14. Nakhleh M K, Jeries R, Gharra A, et al. Detecting active pulmonary tuberculosis with a breath test using nanomaterial-based sensors. Eur Respir J 2014; 43 (5): 1522-5.

15. Coronet Teixeira R, Rodriguez M, Jimenez de Romero N, et al. The potential of a portable, point-of-care electronic nose to diagnose tuberculosis. J Infect 2017; 75(5): 441-7.

16. Suarez-Cuartin G, Giner J, Merino J L, et al. Identification of Pseudomonas aeruginosa and airway bacterial colonization by an electronic nose in bronchiectasis. Respir Med 2018; 136: 111-7.

17. Schnabel R, Fijten R, Smolinska A, et al. Analysis of volatile organic compounds in exhaled breath to diagnose ventilator-associated pneumonia. Sci Rep 2015; 5: 17179.

18. Filipiak W, Beer R, Sponring A, et al. Breath analysis for in vivo detection of pathogens related to ventilator-associated pneumonia in intensive care patients: a prospective pilot study. J Breath Res 2015; 9 (1): 016004.

19. Schnabel R M, Boumans M L, Smolinska A, et al. Electronic nose analysis of exhaled breath to diagnose ventilator-associated pneumonia. Respir Med 2015; 109 (11): 1454-9.

20. Bos L D, Schultz M J, Sterk P J. Exhaled breath profiling for diagnosing acute respiratory distress syndrome. BMC Pulm Med 2014; 14: 72.

21. Bos L D, Weda H, Wang Y, et al. Exhaled breath metabolomics as a noninvasive diagnostic tool for acute respiratory distress syndrome. Eur Respir J 2014; 44 (1): 188-97.

22. Schubert J K, Muller W P, Benzing A, Geiger K. Application of a new method for analysis of exhaled gas in critically ill patients. Intensive Care Med 1998; 24 (5): 415-21.

23. van Geffen W H, Bruins M, Kerstjens H A. Diagnosing viral and bacterial respiratory infections in acute COPD exacerbations by an electronic nose: a pilot study. J Breath Res 2016; 10 (3): 036001.

24. Traxler S, Bischoff A C, Sass R, et al. VOC breath profile in spontaneously breathing awake swine during Influenza A infection. Sci Rep 2018; 8 (1): 14857.

25. Ackermann M, Verleden S E, Kuehnel M, et al. Pulmonary Vascular Endothelialids, Thrombosis, and Angiogenesis in Covid-19. N Engl J Med 2020; 383 (2): 120-8.

26. Force A D T, Ranieri V M, Rubenfeld G D, et al. Acute respiratory distress syndrome: the Berlin Definition. JAMA 2012; 307 (23): 2526-33.

27. Le Gall J R, Lemeshow S, Saulnier F. A new Simplified Acute Physiology Score (SAPS II) based on a European/North American multicenter study. JAMA 1993; 270 (24): 2957-63.

28. Vincent J L, Moreno R, Takata J, et al. The SOFA (Sepsis-related Organ Failure Assessment) score to describe organ dysfunction/failure. On behalf of the Working Group on Sepsis-Related Problems of the European Society of Intensive Care Medicine. Intensive Care Med 1996; 22 (7): 707-10.

29. Trefz P, Pugliese G, Brock B, Schubert J K, Miekisch W. Effects of elevated oxygen levels on VOC analysis by means of PTR-ToF-MS. J Breath Res 2019; 13 (4): 046004.

30. Benjamini Y, Hochberg Y. Controlling the False Discovery Rate: A Practical and Powerful Approach to Multiple Testing. J R Stat Soc Ser B 1995; 57 (1): 289-300.

31. Thevenot E A, Roux A, Xu Y, Ezan E, Junot C. Analysis of the Human Adult Urinary Metabolome Variations with Age, Body Mass Index, and Gender by Implementing a Comprehensive Workflow for Univariate and OPLS Statistical Analyses. J Proteome Res 2015; 14 (8): 3322-35.

32. Zou H, Hastie T. Regularization and variable selection via the elastic net. J R Stat Soc Ser B 2005; 67 (2): 301-20.

33. Breiman L. Random Forests. Machine Learning 2001; 45(1): 5-32.

34. Weston J, Mukherjee S, Chapelle O, Pontil M, Poggio T, Vapnik V. Feature Selection for SVMs. Advances in Neural Information Processing Systems 13 2000.

35. Pihur V, Datta S, Datta S. RankAggreg, an R package for weighted rank aggregation. BMC Bioinformatics 2009; 10: 62.

36. Trefz P, Schmidt M, Oertel P, et al. Continuous real time breath gas monitoring in the clinical environment by proton-transfer-reaction-time-of-flight-mass spectrometry. Anal Chem 2013; 85 (21): 10321-9.

37. Brock B, Kamysek S, Silz J, Trefz P, Schubert J K, Miekisch W. Monitoring of breath VOCs and electrical impedance tomography under pulmonary recruitment in mechanically ventilated patients. J Breath Res 2017; 11 (1): 016005.

38. van de Kant K D, van Berkel J J, Jobsis Q, et al. Exhaled breath profiling in diagnosing wheezy preschool children. Eur Respir J 2013; 41 (1): 183-8.

39. Corradi M, Pignatti P, Manini P, et al. Comparison between exhaled and sputum oxidative stress biomarkers in chronic airway inflammation. Eur Respir J 2004; 24(6): 1011-7.

40. Rahman I. Oxidative stress, chromatin remodeling and gene transcription in inflammation and chronic lung diseases. J Biochem Mol Biol (2003; 36 (1): 95-109.

These documents are referred to hereafter by their number as exponent.

As of Sep. 28, 2020, about 32 millions of people worldwide had been infected with severe acute respiratory syndrome coronavirus 2 (SARS-CoV-2), and about 1 million had died from coronavirus disease 2019 (COVID-19).¹ Approximately 5% of patients with COVID-19 will develop acute respiratory distress syndrome (ARDS), septic shock or multiple organ dysfunction². Around the world, unprecedented research efforts are being focused on the prevention, early detection, diagnosis and management of this lethal disease. To date, only one antiviral drug (remdesivir) has been approved for the treatment of patients hospitalized for COVID-19.³ More recently, a large trial showed that dexamethasone at a daily dose of 6 mg for 10 days substantially reduced the risk of 28 day death (age-adjusted rate ratio [95% confidence interval (CI)]: 0.83 [0.75 to 0.93], particularly in patients with severe disease requiring invasive mechanical ventilation (rate ratio: 0,64 [0.51 to 0.81]).⁴

Although the early immune response may not depend on the severity of the illness, the most severely ill patients show persistent elevations of blood inflammatory markers (such as IL-1α, IL-1β, IL-6, IL-10, IL-18 and TNF-α) 10 days or so after SARS-CoV-2 infection, with a very high risk of subsequent organ injury.⁵⁻⁷ Proteomic and metabolomic studies of serum have described a COVID-19-specific molecular signature; severe and non-severe forms of COVID-19 differ with regard to amino acid metabolism and the expression of acute phase proteins.⁸

Early detection that a person carries or is infected by SARS-CoV-2 and diagnosis of coronavirus disease 2019 (COVID-19) are of the utmost importance but remain challenging.

An object of the invention is to contribute to the screening, diagnosis, or monitoring of the presence of this virus and associated disease in human.

To this aim, the invention provides a method for analysis, the method comprising:
obtaining a sample comprising elements coming from breath exhaled from a person;
exposing the sample to a spectrometer;
determining at least one value of a signal intensity and/or concentration of ions defined by its mass-to-charge ratio (m/z) given by the spectrometer in at least one range,
the or each range being defined by a median value and limits of the range;
applying at least one test to the value,
the range(s) and the test being configured for identifying that:
   a person carries and/or is infected with SARS-CoV-2,
   a person does not carry and/or is not infected with SARS-CoV-2,
   a person suffers from COVID-19, and/or
   a person does not suffer from COVID-19; and
communicating a message according to a result of the test or decision rule.

In one embodiment, the step of determining comprises determining only one value of signal intensity and/or concentration of ions defined by its mass-to-charge ratio (m/z) given by the spectrometer in only one range.

In one embodiment, the range comprises m/z=99.08 (99.04-99.14), the method being performed preferably for identifying that a person carries or is infected by SARS-CoV-2 or that a person does not carry or is not infected by SARS-CoV-2.

In another embodiment, the step of determining comprises determining values of signal intensities and/or concentrations of ions defined by their mass-to-charge ratios (m/z) given by the spectrometer in a group of ranges.

In one embodiment, the range comprises one of the following ranges or the group comprises at least one of the following ranges, preferably all of them, and for example consists in all of them:
m/z=135.09 (135.03-135.17),
m/z=143.15 (143.07-143.22),
m/z=99.08 (99.04-99.14), and
m/z=111.12 (111.07-111.18).

In another embodiment, the range comprises one of the following ranges or the group comprises at least one of the following ranges, preferably all of them, and for example consists in all of them:
m/z=135.09 (135.03-135.17),
m/z=143.15 (143.07-143.22),
m/z=99.08 (99.04-99.14),
m/z=111.12 (111.07-111.18),
m/z=71.05 (71.02-71.09),
m/z=55.05 (55.03-55.09),
m/z=83.09 (83.05-83.13), and
m/z=93.04 (93.00-93.09).

In another embodiment, the range comprises one of the following ranges or the group comprises at least one of the ranges of the following table, preferably all of them, and for example consists in all of them:

| (m/z [M + H]⁺) | (m/z [M + H]⁺) | (m/z [M + H]⁺) |
|---|---|---|
| median | lower limit | upper limit |
| 135.09 | 135.03 | 135.17 |
| 143.15 | 143.07 | 143.22 |
| 99.08 | 99.04 | 99.14 |
| 111.12 | 111.07 | 111.18 |
| 71.05 | 71.02 | 71.08 |
| 55.05 | 55.03 | 55.09 |
| 83.09 | 83.05 | 83.13 |
| 93.04 | 93.00 | 93.09 |
| 29.01 | 29.00 | 29.03 |
| 115.11 | 115.05 | 115.18 |
| 97.07 | 97.02 | 97.10 |
| 63.01 | 62.98 | 63.04 |
| 47.05 | 47.03 | 47.08 |
| 65.06 | 65.03 | 65.10 |
| 179.15 | 179.07 | 179.25 |
| 73.07 | 73.04 | 73.11 |
| 30.98 | 30.97 | 31.00 |
| 79.06 | 79.02 | 79.10 |
| 81.07 | 81.04 | 81.12 |
| 38.03 | 38.02 | 38.06 |
| 39.02 | 39.01 | 39.04 |
| 53.04 | 53.02 | 53.07 |
| 85.10 | 85.07 | 85.15 |
| 81.02 | 80.98 | 81.06 |
| 41.00 | 40.98 | 41.03 |
| 117.09 | 117.04 | 117.16 |
| 43.99 | 43.97 | 44.02 |
| 31.00 | 30.99 | 31.02 |
| 71.09 | 71.06 | 71.13 |
| 45.03 | 45.01 | 45.06 |
| 43.97 | 43.94 | 43.99 |
| 89.06 | 89.02 | 89.11 |
| 66.96 | 66.93 | 67.00 |
| 107.09 | 107.03 | 107.15 |
| 91.06 | 91.02 | 91.11 |
| 53.00 | 52.97 | 53.03 |
| 75.05 | 75.01 | 75.09 |
| 27.00 | 26.98 | 27.01 |
| 31.02 | 31.00 | 31.03 |
| 67.06 | 67.03 | 67.09 |
| 60.05 | 60.03 | 60.09 |
| 45.00 | 44.98 | 45.02 |
| 39.03 | 39.01 | 39.05 |
| 27.01 | 26.99 | 27.03 |
| 33.03 | 33.02 | 33.05 |
| 41.04 | 41.02 | 41.06 |
| 29.00 | 28.99 | 29.02 |
| 77.06 | 77.03 | 77.10 |
| 31.05 | 31.04 | 31.07 |
| 61.04 | 61.00 | 61.07 |
| 28.00 | 27.98 | 28.01 |
| 57.03 | 57.01 | 57.06 |
| 137.13 | 137.07 | 137.21 |
| 57.07 | 57.05 | 57.10 |
| 62.03 | 62.00 | 62.06 |
| 68.06 | 68.04 | 68.10 |
| 27.02 | 27.01 | 27.04 |
| 43.06 | 43.04 | 43.08 |
| 87.08 | 87.04 | 87.13 |
| 47.01 | 46.99 | 47.04 |
| 52.96 | 52.94 | 52.99 |
| 66.05 | 66.02 | 66.08 |
| 51.04 | 51.02 | 51.07 |
| 69.07 | 69.05 | 69.11 |
| 29.04 | 29.02 | 29.05 |

In one embodiment, the method takes into account at least one of the following elements:
a symptom score,
whether the person had a corticosteroid therapy before sampling, and
whether the person had oxygenotherapy before sampling.

The invention relates to the metabolomics of exhaled breath and provides a SARS-CoV-2 and/or COVID-19-specific breath metabolomic signature.

It concerns for example the persons carrying SARS-CoV-2 or those infected with SARS-CoV-2.

It also concerns for example COVID-19 patients, among others critically ill COVID-19 patients, for example those requiring invasive mechanical ventilation.

In instant application, the persons who carry SARS-CoV-2 comprise infected people (COVID-19 disease) and persons who do not suffer from COVID-19 (asymptomatic people).

This signature is formed for example by a set of 65 volatile organic compounds. Ranking aggregation showed that the eight most prominent volatile organic compounds in this signature are m/z=135.09, m/z=143.15, m/z=99.08, m/z=111.12, m/z=71.05, m/z=55.05, m/z=83.09 and, m/z=93.04, by order of importance and as defined by their mass-to-charge ratio ([M+H]⁺) with PTR-MS detection. (“PTR-MS” stands for proton transfer reaction mass spectrometry.)

Using the whole signature, we can differentiate between COVID-19 and non-COVID-19 patients with an accuracy of 93% (sensitivity: 90%, specificity: 94%, area under the receiver operating characteristic curve: 0.93-0.98).

With the eight most prominent compounds, we can differentiate between COVID-19 and non-COVID-19 patients with an accuracy of 90-93% (sensitivity: 85-90%, specificity: 93-94%, area under the receiver operating characteristic curve: 0.95-0.98).

With the four most important features (m/z=99.08, m/z=111.12, m/z=135.09 and m/z=143.15, as defined by their mass-to-charge ratio ([M+H]+) with PTR-MS detection), we can differentiate between COVID-19 and non-COVID-19 patients with an accuracy of 89-93% (sensitivity: 90-98%, specificity: 88-94%, area under the receiver operating characteristic curve: 0.89-0.95). These four most important features also discriminate the two groups (COVID-19 positive and COVID-19 negative) in the longitudinal modelling of the first 10 days of hospitalization.

But this signature may be formed in another example by only one volatile organic compound. Indeed, with only one feature such as m/z=99.08 in combination with the following elements:
a symptom score,
whether the person had a corticosteroid therapy before sampling, and
whether the person had oxygenotherapy before sampling,
we can differentiate between persons who carry/are infected with the virus and those who are not (area under the receiver operating characteristic curve: 0.85).

Thus, non-invasive detection of volatile organic compounds in exhaled breath may identify patients with COVID-19 and provide a diagnosis of COVID-19. It also permits to identify the persons who carry or are infected by the virus, with or without clinical symptoms. It also permits to identify the persons who do not carry or are not infected by the virus, and therefore allows to exclude the diagnosis of COVID-19. The knowledge of this specific breathprint enables the development of rapid, non-invasive, point-of-care tests for large-scale COVID-19 screening or monitoring disease severity and control.

Thus, in one embodiment, the method is a method for the diagnosis of COVID-19.

It cannot be excluded that the invention contributes not only to the diagnosis, but also to the prognosis/evaluation of the severity and specific impairment of certain organs as well as to the evaluation of the effect of the treatments administered.

Different kinds of tests or decision rules may be used such as the algorithms known as elastic net, random forest or support vector machine, as explained below.

In one embodiment, the determining step comprises determining whether one or at least two, for example three, four, five, six or seven of the components are present in the sample.

In one embodiment, the spectrometer is a mass spectrometer, for example a proton transfer reaction mass spectrometer.

Using a proton transfer reaction mass spectrometer enables to have an immediate analysis of the sample and an immediate result. But other types of mass spectrometers (other than PTR-MS) would allow the detection of the same compounds.

Similarly, the method may be performed according to different modalities, for example not by directly analyzing the breath exhaled by the person or the patient (either by direct connection to a ventilator or by using a device for breath collection for breath analysis such as the Buffered End-Tidal Breath Sampling Inlet (BET-med) device (Ionicon, Innsbruck, Austria) but by adding a storage step of the sample: the exhaled breath is taken from the patient's bed and stored in bags (Tedlar bags type) or on desorption tubes (Tenax type) which are then analyzed in another place (e.g. laboratory)) with identical (PTR-MS) or similar technologies (GC-MS, GC-IMS . . . ).

The method of the invention may also have at least one of the following features:
the person is undergoing invasive mechanical ventilation;
the sample is a sample of the exhaled breath;
the step of obtaining a sample comprises obtaining the sample via a transfer line in direct communication with the person, for example the transfer line being connected to an end of an endotracheal tube installed on the person.
it comprises heating the transfer line; and
the person is inspiring air, a mixture of nitrogen and oxygen or pure oxygen;

The invention also provides a device for the diagnosis, screening or monitoring of SARS-CoV-2 and/or COVID-19, the device comprising:
means for determining at least one value of a signal intensity and/or concentration of ions defined by its mass-to-charge ratio (m/z) given by the spectrometer in at least one range,
the or each range being defined by a median value and limits of the range;
means for applying at least one test to the value,
the range(s) and the test being configured for identifying that:
   a person carries and/or is infected with SARS-CoV-2,
   a person does not carry and/or is not infected with SARS-CoV-2,
   a person suffers from COVID-19, and/or
   a person does not suffer from COVID-19.

The device of the invention may also comprise at least one of the following features:
means for receiving the breath sample;
a transfer line having a connector configured for connecting to an end of an endotracheal tube;
means for heating the transfer line; and
a spectrometer, such as a mass spectrometer, for example a proton-transfer-reaction mass spectrometer;

The invention also provides a program comprising code instructions configured for controlling an execution of the method of the invention or for controlling a device according to the invention, as well as a process for providing this program on a communication network in view of downloading the program.

### PRESENTATION OF THE FIGURES

We will now present two non-limiting examples embodying the invention with the help of the following figures on which:

FIGS. 1 to 6 show graphs illustrating the results of analysis of components in the breath exhaled from patients suffering from Covid-19 in a first example embodying the invention;

FIG. 7 shows an embodiment of a device according to the invention;

FIG. 8 shows graphs obtained when performing the method of the invention with this device;

FIG. 9 shows graphs obtained for the analysis of the four most prominent features (m/z=99.08, m/z=111.12, m/z=135.09 and m/z=143.15, as defined by their mass-to-charge ratio ([M+H]⁺)) with proton transfer reaction-mass spectrometry;

FIG. 10 illustrates one of the tests or decision rules used in the embodiment of the method of the invention;

FIG. 11 is a graph representing the level of expression of the m/z 99.08 signal according to the groups of persons in a second example embodying the invention;

FIG. 12 is a graph giving more details about the “No infection group” of FIG. 11;

FIG. 13 is a graph confronting the m/z 99.08 signal intensity with the Ct (cycle threshold) in the second example;

FIG. 14 is a graph confronting the m/z 99.08 signal intensity with the time elapsed between the PCR being performed in the second example;

FIGS. 15 and 16 are a graph and a matrix showing diagnostic performances in this example; and

FIGS. 17 and 18 are similar graph and matrix about a complementary analysis in this example.

### DETAILED DESCRIPTION

### First Example

Breath analysis is an innovative, non-invasive, technique for detecting volatile organic compounds (VOCs) with potential for use in diagnosis and large-scale screening.^{9,10} Thousands of VOCs have been identified in human breath following infectious, inflammatory or pathological events.^{11,12} The analysis of exhaled breath can be used to diagnose tuberculosis, invasive fungal infections, and bacterial colonization of the respiratory tract¹³⁻¹⁶, together with ARDS and ventilator-associated pneumonia in patients in an intensive care unit (ICU).¹⁷⁻²² Likewise, VOC analysis is of value in the diagnosis of viral infections in patients with chronic obstructive pulmonary disease and of influenza infections in a swine model.^{23,24}

We used real-time, online, proton transfer reaction time-of-flight mass spectrometry to perform a metabolomic analysis of exhaled breath from adults undergoing invasive mechanical ventilation in an intensive care unit due to severe COVID-19 or non-COVID-19 acute respiratory distress syndrome (ARDS).

Between March 25th and Jun. 25, 2020, we included 40 patients with ARDS, of whom 28 had proven COVID-19. In a multivariate analysis, we identified a characteristic breathprint for COVID-19. This signature is formed by a set of 65 volatile organic compounds. Ranking aggregation showed that the eight most prominent volatile organic compounds in this signature are m/z=135.09, m/z=143.15, m/z=99.08, m/z=111.12, m/z=71.05, m/z=55.05, m/z=83.09 and, m/z=93.04, by order of importance and as defined by their mass-to-charge ratio ([M+H]⁺) with PTR-MS detection. Using the whole signature, we can differentiate between COVID-19 and non-COVID-19 patients with an accuracy of 93% (sensitivity: 90%, specificity: 94%, area under the receiver operating characteristic curve: 0.93-0.98). With the eight most prominent compounds, we can differentiate between COVID-19 and non-COVID-19 patients with an accuracy of 90-93% (sensitivity: 85-90%, specificity: 93-94%, area under the receiver operating characteristic curve: 0.95-0.98). With the four most important features (m/z=99.08, m/z=111.12, m/z=135.09 and m/z=143.15, as defined by their mass-to-charge ratio ([M+H]+) with PTR-MS detection), we can differentiate between COVID-19 and non-COVID-19 patients with an accuracy of 89-93% (sensitivity: 90-98%, specificity: 88-94%, area under the receiver operating characteristic curve: 0.89-0.95).

The real-time, non-invasive detection of volatile organic compounds in exhaled breath permits to identify patients with COVID-19.

### Study Design, Oversight and Participants

This study was conducted at the ICU of Raymond Poincaré Hospital (Garches, France). Adult patients (aged 18 or over) in ICUs were eligible for inclusion if they had ARDS and required invasive mechanical ventilation. ARDS was defined as all of the following: (i) acute onset, i.e. within one week of an apparent clinical insult, followed by progression of the respiratory syndrome, (ii) bilateral opacities on chest imaging not explained by another lung disease (e.g. pleural effusion, atelectasis, nodules etc.), (iii) no evidence of heart failure or volume overload, and (iv) PaO₂/FiO₂≤300 mm Hg, and positive end expiratory pressure (PEEP)≤5 cm H₂O.²⁶ The main exclusion criteria were pregnancy, an expectation of death within 48 hours, and the withholding or withdrawal of treatment.

### Study Measurements and Procedures

Variables recorded at baseline were patient demographics and anthropometrics, the source of infection and the severity of illness (according to the Simplified Acute Physiology Score (SAPS) II and the Sequential Organ Failure Assessment (SOFA)).^{27,28} The following variables were recorded at baseline and daily during the hospital stay: core body temperature, vital signs, central hemodynamic data, standard laboratory data, microbiological and virologic data. Samples of blood and nasopharyngeal, bronchial or bronchoalveolar lavage fluids were assayed for SARS-CoV-2 and other respiratory viruses, using a PCR test. Were also recorded life-supportive therapies including mechanical ventilation, renal replacement therapy, intravenous fluids bolus and the administration of vasopressors, and adjunct therapies including corticosteroids, thiamine, vitamin C, other vitamins, nutritional supplements, blood products, anticoagulants, sedatives, stress ulcer prophylaxis and anti-infective drugs.

### Breath Analysis

Each patient's exhaled breath was analyzed daily in the morning until discharge. Measurements were made with a proton-transfer-reaction quadrupole time-of-flight mass spectrometer (PTR-Qi-TOF MS) (Ionicon Analytik GmbH, Innsbruck, Austria) placed outside the patient room. Samples were obtained via a heated transfer line (length: 1.6 m) connected directly to the end of the endotracheal tube (i.e. without disconnection from the mechanical ventilator) and with an air flow of 50 mL/min. To eliminate the dependency on the oxygen concentration in the sample matrix, recordings were performed in patients with a fraction of inspired oxygen of 100% for at least 3 mins.²⁹ The acquisition took 2 mins. H₃O⁺ was used as the primary ion and the instrument settings were as follows: source voltage, 120 V; drift tube pressure, 3.8 mbar; drift tube temperature, 60° C.; and drift tube voltage, 959 V. The mass spectrum was acquired up to m/z=392, with a time resolution of 0.1 s.

We recall that, when presenting data of a spectrometer, it is common to use the (officially) dimensionless m/z. This quantity, although it is informally called the mass-to-charge ratio, more accurately speaking represents the ratio of the mass number m and the charge number, z. A spectrometer can measure this mass-to-charge ratio of ionized compounds. For example for a molecule M, the form [M+H]⁺ may be detected with PTR-MS when using H₃O⁺ as the primary ion.

### Data and Statistical Analysis

Patient characteristics were expressed as the median [interquartile range (IQR)] for continuous variables and the frequency (percentage) for categorical variables. Patients with and without COVID-19 were compared using Fisher's exact test for categorical variables, and a t-test or the Mann-Whitney test for normally and non-normally distributed continuous variables (as evaluated with the d'Agostino-Pearson test), respectively.

Mass spectrometry data were processed with the ptairMS software developed by CEA under the CeCILL licence (https://github.com/camilleroquencourt/ptairMS) and included mass calibration, expiratory phase detection, peak detection and quantification, normalization, alignment, isotope identification and the imputation of missing values. After aligning each individual peak, ions detected in more than 70% of at least one group (COVID vs. non-COVID-19) were kept; this resulted in 81 features. Missing values (corresponding to ions in exhaled breath that were not detected by the preprocessing algorithm) were imputed with the ptairMS package, which returns to the raw data and integrates the noise at the exact missing m/z. Data were then log2-transformed and standardized. Outliers (patients with a z-score>3 for at least five features) were deleted. In the remaining patients, saturated ions (acetone, H₃O⁺, H₂O—H₃O⁺, oxygen) and isotopes were deleted to leave a final table of 65 features.

For the univariate analysis, a Wilcoxon test was performed and p-values were adjusted to control for the false discovery rate.³⁰ For multivariate analysis, data were first analyzed with unsupervised learning principal component analysis and then with supervised machine learning algorithms (orthogonal partial least-squares discriminant analysis, linear support vector machine, elastic net, and random forest) with the R packages ropis, e1071, glmnet, and randomForest.³¹⁻³⁴ A 10-fold cross-validation was repeated four times, in order to avoid overfitting the small number of data points. The model's parameters were tuned to optimize the cross-validation accuracy.

To rank the features in order of importance, we used rank aggregation (with RankAggreg R package³⁵) of the ordered p-values from the Wilcoxon test, the rank in decreasing order of absolute value for the loadings in the principal component analysis, the variable importance in projection from the orthogonal partial least-squares discriminant analysis, the coefficient values from the elastic net and the support vector machine, and the feature importance from the random forest. The effects of tidal volume, PEEP, respiratory rate, serum C-reactive protein (CRP) level, body temperature, and renal replacement were also investigated in a correlation test; the secondary principal component in the principal component analysis was used to determine whether or not these covariates were confounding factors (using Pearson's test for continuous variables and a chi-squared test for categorical variables).

Longitudinal analysis of the most important features was performed with a mixed effects model, using a sum of 4 spline functions uniformly distributed over time for the fixed effect. Intergroup differences in trends and mean concentrations were assessed with an F-test (p-value<0.05) adjusted to correct for the false discovery rate. Correlations between VOC concentrations, the SAPS II, the SOFA score and the viral load were analyzed using Pearson's correlation test, with correction for the false discovery rate.

### Results

### Patients

Between March 25th and Jun. 25, 2020, 40 patients (of whom 28 had confirmed COVID-19-related ARDS) were included in the study and a total of 303 measurements were made.

Compared with the patients with non-COVID-19 ARDS, the patients with COVID-19 ARDS had (i) a higher incidence of treatment with glucocorticoids prior to admission, (ii) a higher respiratory rate, FiO₂, PEEP and CRP on admission, (iii) a higher incidence of treatment with hydroxychloroquine and fludrocortisone after admission, and (iv) a greater likelihood of renal replacement therapy (Table 1).

TABLE 1
Patient characteristics and treatments

| | COVID-19 | | Non-COVID-19 | | p |
|---|---|---|---|---|---|
| | ARDS | | ARDS | | value |
| Number of | 28 | | 12 | | — |
| patients (n) | | | | | |
| Males/females (n) | 20/8 | | 6/6 | | 0.28 |
| Age (years) | 61 | [55-72] | 72 | [54-79] | 0.75 |
| Body weight (kg) | 80.0 | [66.6-87.6] | 86.5 | [65.3-94.1] | 0.71 |
| Height (cm) | 170 | [164-175] | 173 | [169-175] | 0.55 |
| Body mass index | 26.3 | [23.7-32.4] | 28.9 | [23.0-30.9] | 0.79 |
| (kg/m²) | | | | | |
| SAPS II score in the | 62 | [49-68] | 46 | [40-57] | 0.051 |
| first 24 hours | | | | | |
| SOFA score in the | 11 | [7-12] | 8 | [5-12] | 0.37 |
| first 24 hours | | | | | |
| Comorbidities: | | | | | |
| (n (%)) | | | | | |
| high blood pressure | 11 | (39) | 6 | (50) | 0.73 |
| chronic obstructive | 2 | (7) | 1 | (8) | >0.99 |
| pulmonary disease | | | | | |
| ischemic cardiac | 5 | (18) | 3 | (25) | 0.68 |
| disease | | | | | |
| cancer | 2 | (7) | 3 | (25) | 0.15 |
| Treatments before | | | | | |
| admission: (n (%)) | | | | | |
| glucocorticoids | 0 | (0) | 3 | (25) | 0.022 |
| conversion enzyme | 5 | (18) | 1 | (8) | 0.54 |
| inhibitors | | | | | |
| angiotensin | 2 | (7) | 2 | (16) | 0.57 |
| antagonists | | | | | |
| Interventions after | | | | | |
| admission: (n (%)) | | | | | |
| catecholamines | 17 | (61) | 4 | (33) | 0.17 |
| renal replacement | 9 | (32) | 0 | (0) | 0.038 |
| therapy | | | | | |
| Treatments after | | | | | |
| admission: (n (%)) | | | | | |
| hydroxychloroquine | 27 | (96) | 1 | (8) | <0.0001 |
| remdesivir | 2 | (7) | 0 | (0) | >0.99 |
| lopinavir/ritonavir | 7 | (25) | 0 | (0) | 0.081 |
| glucocorticoids | 11 | (39) | 6 | (50) | 0.73 |
| fludrocortisone | 1 | (4) | 4 | (33) | 0.022 |
| eculizumab | 12 | (43) | 4 | (33) | 0.73 |
| Body temperature at | 37.4 | [36.5-38.3] | 37.3 | [36.8-37.8] | 0.84 |
| first sample (° C.) | | | | | |
| Respiratory rate at | 26 | [25-28] | 20 | [18-23] | <0.0001 |
| first sample | | | | | |
| (breaths per min) | | | | | |
| Tidal volume at first | 420 | [400-475] | 438 | [400-490] | 0.99 |
| sample (mL) | | | | | |
| Fraction of inspired | 80 | [50-100] | 48 | [31-68] | 0.007 |
| oxygen at first | | | | | |
| sample (%) | | | | | |
| Positive end- | 10 | [8-13] | 5.5 | [5-8] | 0.0002 |
| expiratory pressure | | | | | |
| at first | | | | | |
| sample (cm H₂O) | | | | | |
| Serum creatinine at | 74 | [56-137] | 67 | [44-86] | 0.30 |
| first sample (μM) | | | | | |
| Serum C-reactive | 195 | [175-268] | 76 | [23-119] | 0.002 |
| protein at first | | | | | |
| sample (mg/L) | | | | | |

Continuous data are presented as the median [IQR].

### Metabolomic Analysis of Exhaled Breath

We first used an untargeted metabolomic strategy to identify biomarkers associated with COVID-19 ARDS. To this end, we used the first breath sample collected after admission. Twelve of the 40 participants had been hospitalized for more than 10 days at the start of the sampling period and so were excluded from this first part of the study. Hence, we analyzed 18 patients with COVID-19 ARDS and 10 with non-COVID-19 ARDS.

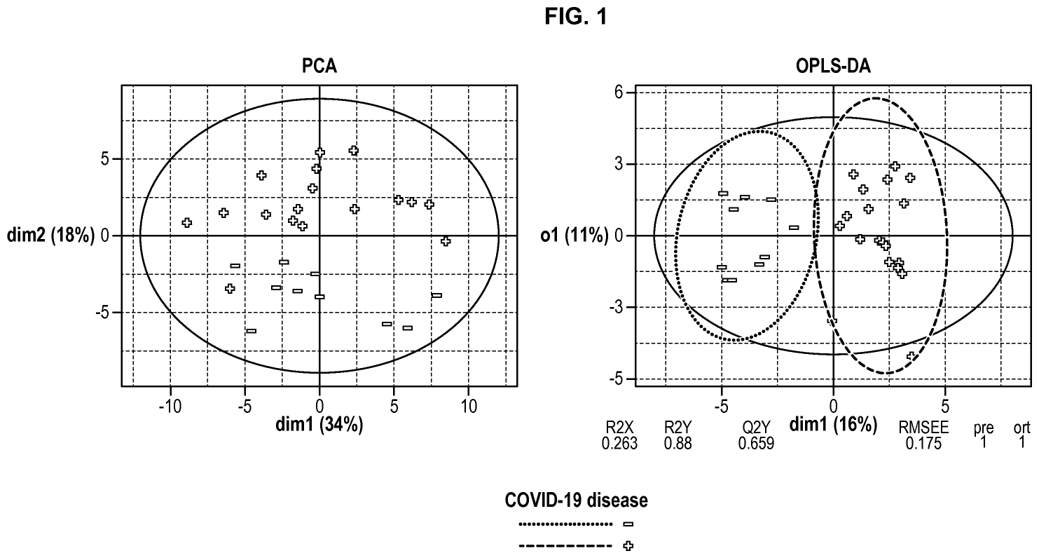

A principal component analysis and an orthogonal partial least-squares discriminant analysis showed that COVID-19 was associated with a specific signature in the exhaled breath, i.e. the breathprint could discriminate between COVID-19 and non-COVID-19 patients. FIG. 1 shows this unsupervised analysis. A primary component analysis (left) and an orthogonal primary least-squares discriminant analysis (right) of the breath signature in intubated, mechanically ventilated ICU patients with a positive (red) or negative (blue) PCR test for SARS-CoV-2.

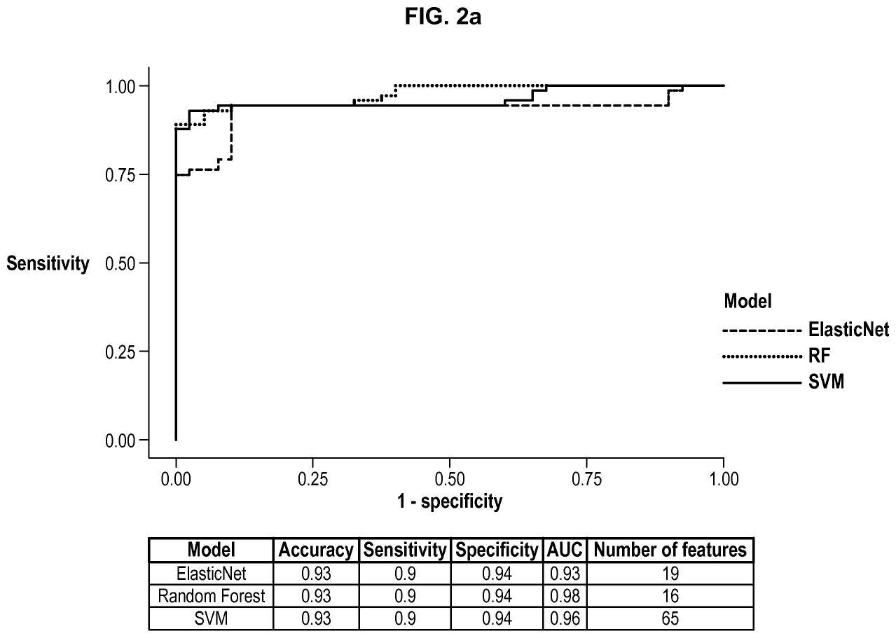

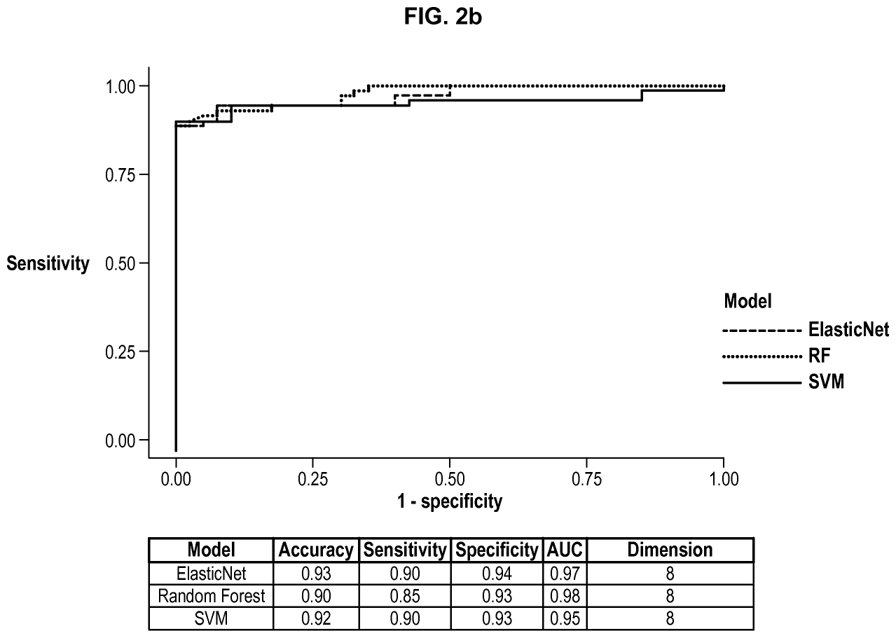

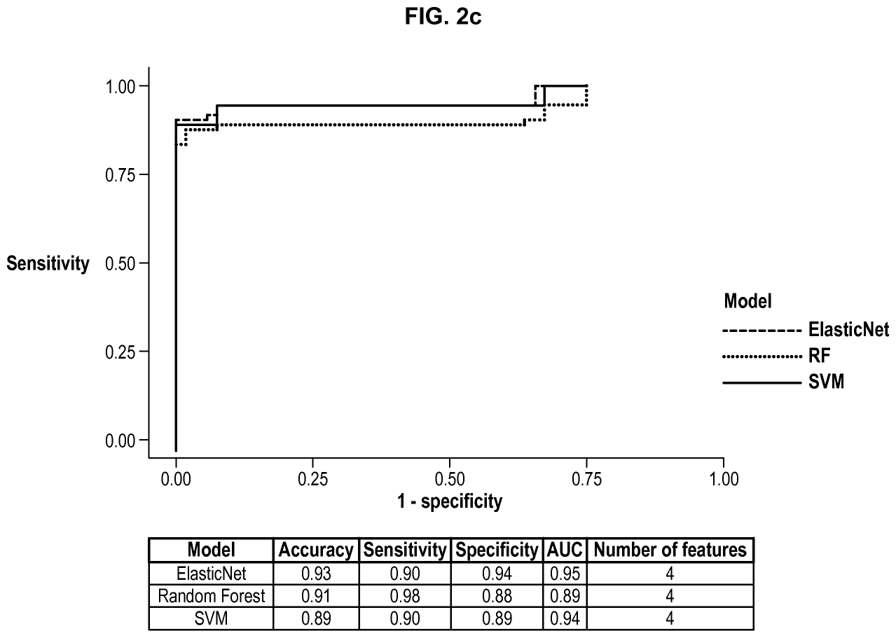

The use of three machine learning classification algorithms (elastic net, support vector machine and random forest) yielded an accuracy of up to 93%, with a 10-fold cross validation repeated 4 times, using 19 (elastic net), 16 (random forest) or 65 (support vector machine) features from the 65 features of the whole signature. The corresponding receiver operating characteristic curves are shown in FIG. 2a. These curves are for models classifying patients with COVID-19 vs. non-COVID-19 ARDS. After internal cross-validation, the sensitivity was 90% and the specificity was 94%.

In order to identify possible confounding effects, we first tested the relationship between the COVID-19 status and all the available covariables for patient demographics (sex, age, body weight, height, and body mass index), clinical and biological data (body temperature, SAPS II and SOFA scores, serum CRP and serum creatinine), comorbidities (high blood pressure, chronic obstructive pulmonary disease, ischemic cardiac disease and cancer), ventilation parameters (respiratory rate, positive end-expiratory pressure and tidal volume) and treatments (catecholamines, renal replacement, hydroxychloroquine, remdesivir, lopinavir/ritonavir, glucocorticoids, fludrocortisone and eculizumab). We applied a Wilcoxon test to quantitative variables and a chi-squared test to qualitative variables, with false discovery rate correction in both cases.

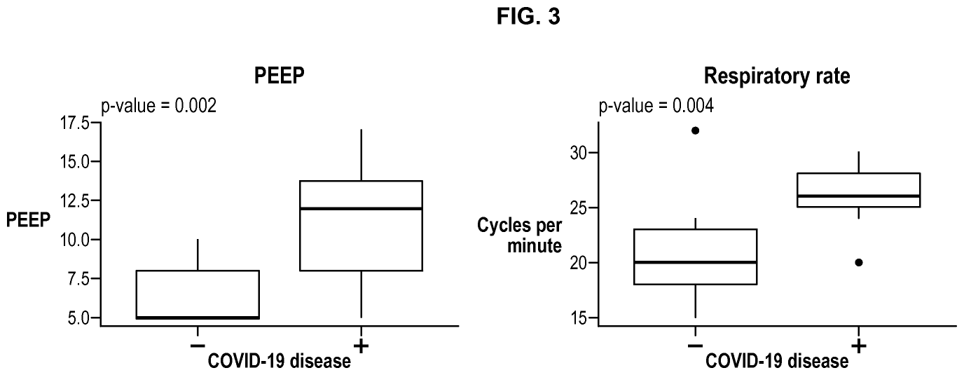

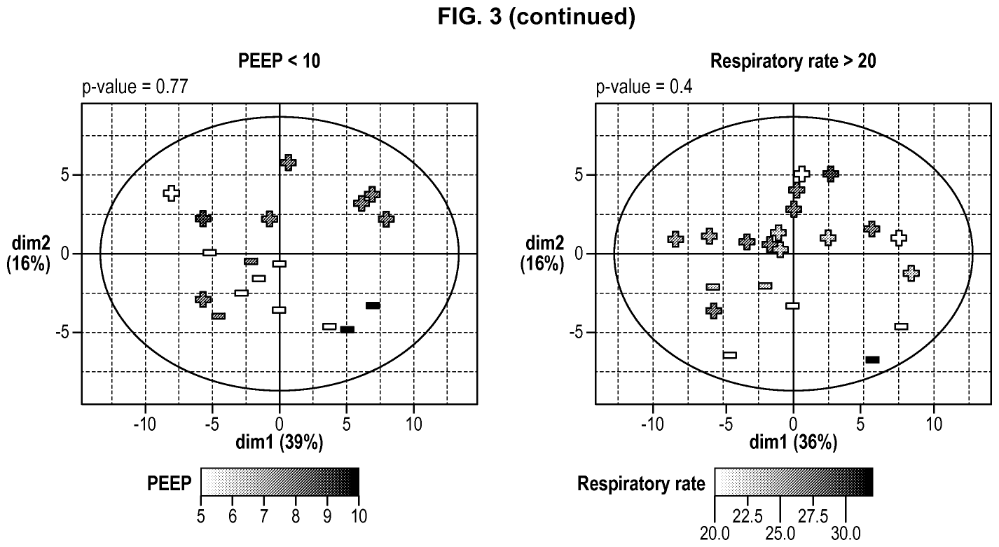

The variables significantly related to COVID-19 status (p-value<0.05) were the positive end-expiratory pressure and the respiratory rate (FIG. 3-A). FIG. 3 shows the effect of the positive end-expiratory pressure and respiratory rate as follows:

A. The positive end-expiratory pressure (PEEP) and respiratory rate, according to COVID-19 ARDS status. The p-value from the Wilcoxon test is indicated.

B. A principal component analysis of the subsets corresponding to patients with PEEP<10 (left) or a respiratory rate>20 (right).

The “+” and “−” symbols refer to the patient's COVID-19 status. The p-value from the Pearson correlation test comparing the factor intensities (displayed as a color gradient) and the scores in the secondary component of the principal component analysis (i.e. the component that best discriminated between COVID-19 ARDS and non-COVID-19 ARDS) is shown.

To determine whether these variables had a confounding effect, we therefore restricted the sample to two rebalanced groups by keeping only patients with a positive end-expiratory pressure<10 on one hand or a respiratory rate>20 on the other. We then applied Pearson's correlation test to the secondary component from the principal component analysis of this restricted dataset. We observed that in both cases the secondary component still distinguished between cases of COVID-19 ARDS and non-COVID-19 ARDS disease and that there was no correlation with the positive end-expiratory pressure or the respiratory rate (FIG. 3-B).

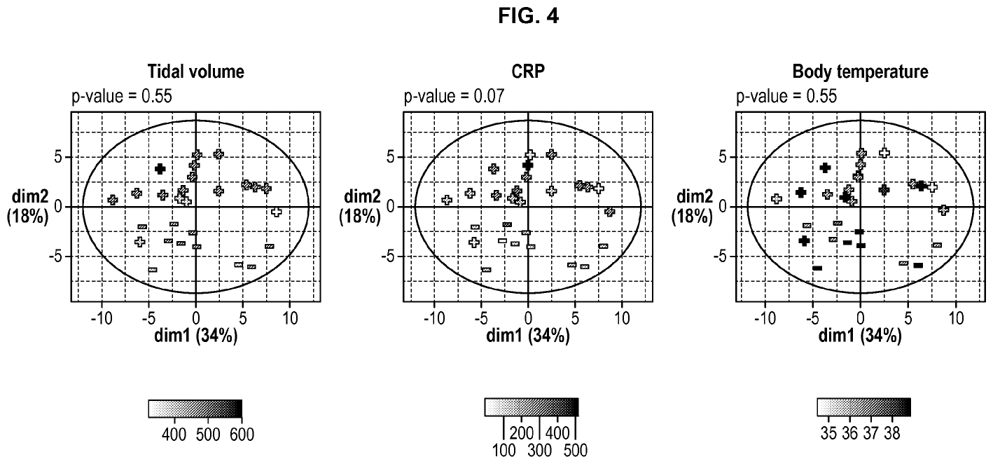

We used the same procedure for the other covariables with the complete dataset. None of them was significantly correlated with the secondary component of the principal component analysis (FIG. 4). FIG. 4 shows the effect of tidal volume, CRP and body temperature in a principal component analysis and a correlation analysis. COVID-19 status is represented by the “+” and “−”, whereas the color scale represents the factor's intensity. The p-value of the Pearson correlation test comparing the secondary component and the corresponding factor is shown on each graph.

To determine which VOCs were most discriminant for COVID-19 status, we performed rank aggregation using the various metrics from the previously mentioned models and the hypothesis tests.

The eight most prominent volatile organic compounds in this signature were m/z=135.09, m/z=143.15, m/z=99.08, m/z=111.12, m/z=71.05, m/z=55.05, m/z=83.09 and, m/z=93.04, by order of importance and as defined by their mass-to-charge ratio ([M+H]⁺) with PTR-MS detection. With these eight most prominent compounds, we can differentiate between COVID-19 and non-COVID-19 patients with an accuracy of 90-93% (sensitivity: 85-90%, specificity: 93-94%, area under the receiver operating characteristic curve: 0.95-0.98).

With the four most important features (m/z=99.08, m/z=111.12, m/z=135.09 and m/z=143.15, as defined by their mass-to-charge ratio ([M+H]⁺) with PTR-MS detection), we can differentiate between COVID-19 and non-COVID-19 patients with an accuracy of 89-93% (sensitivity: 90-98%, specificity: 88-94%, area under the receiver operating characteristic curve: 0.89-0.95).

The corresponding receiver operating characteristic curves are shown in FIG. 2b for the eight most prominent compounds (m/z=135.09, m/z=143.15, m/z=99.08, m/z=111.12, m/z=71.05, m/z=55.05, m/z=83.09 and, m/z=93.04) and FIG. 2c for the four most prominent compounds (m/z=135.09, m/z=143.15, m/z=99.08 and m/z=111.12).

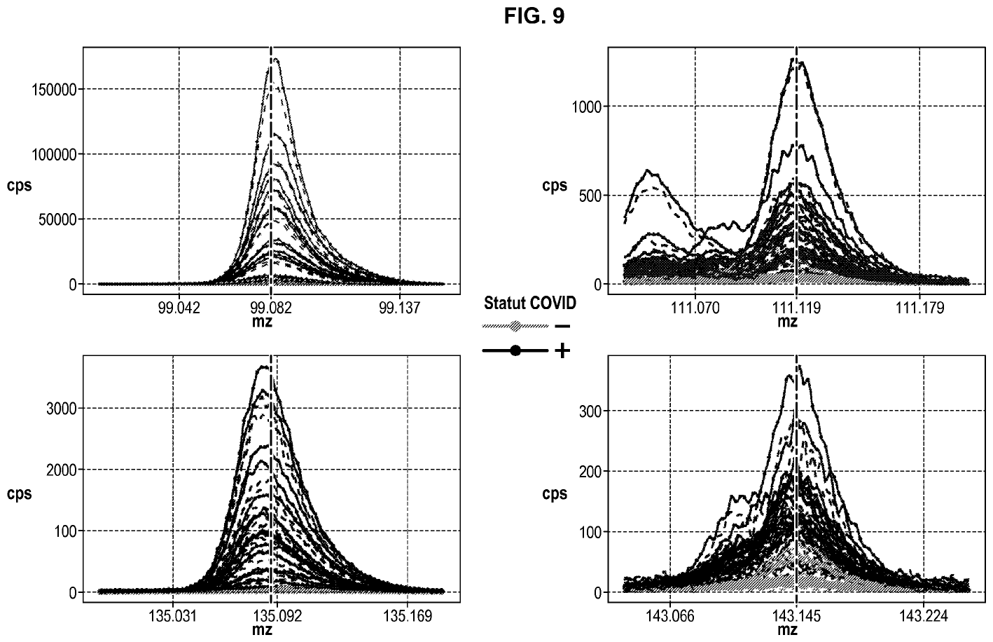

FIG. 9 shows graphs obtained for the analysis of the four most prominent features (m/z=99.08, m/z=111.12, m/z=135.09 and m/z=143.15, as defined by their mass-to-charge ratio ([M+H]⁺)) with proton transfer reaction—mass spectrometry. Each curve represents a patient of the study. And the curves of the patients having a COVID+status and a COVID-status are visually differentiated. The range associated with each median value is illustrated.

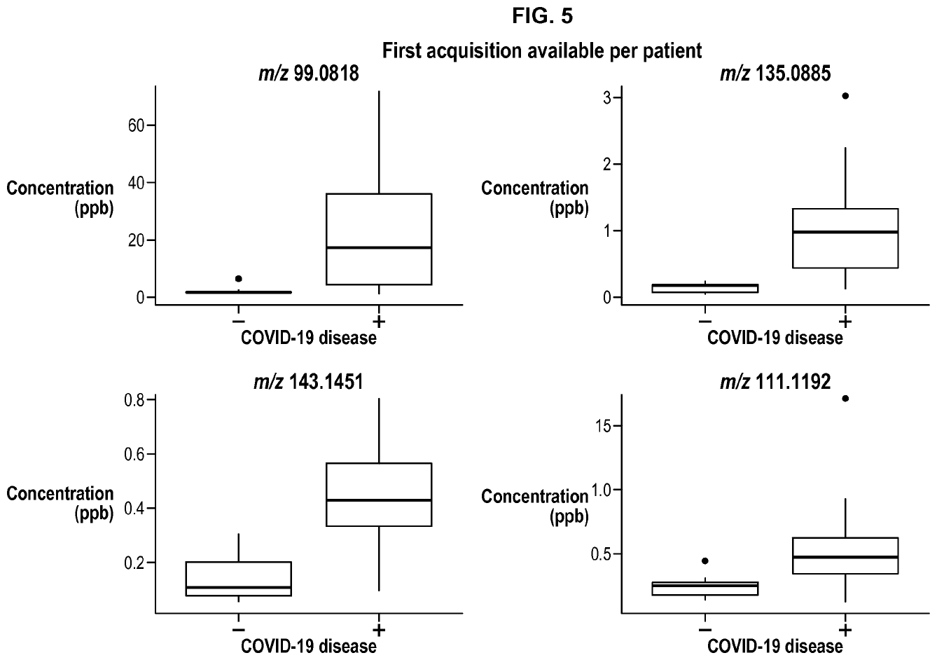

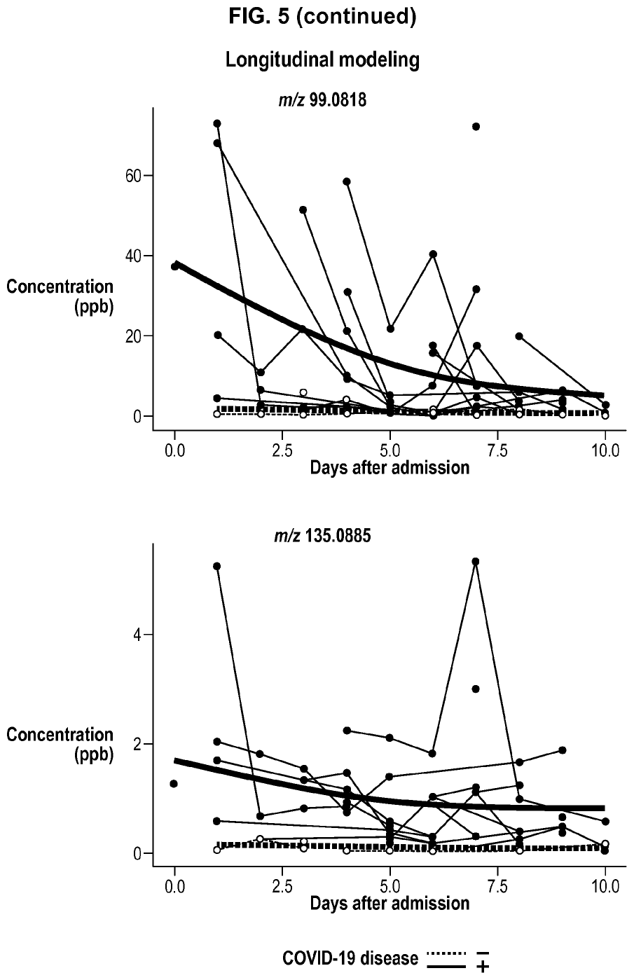

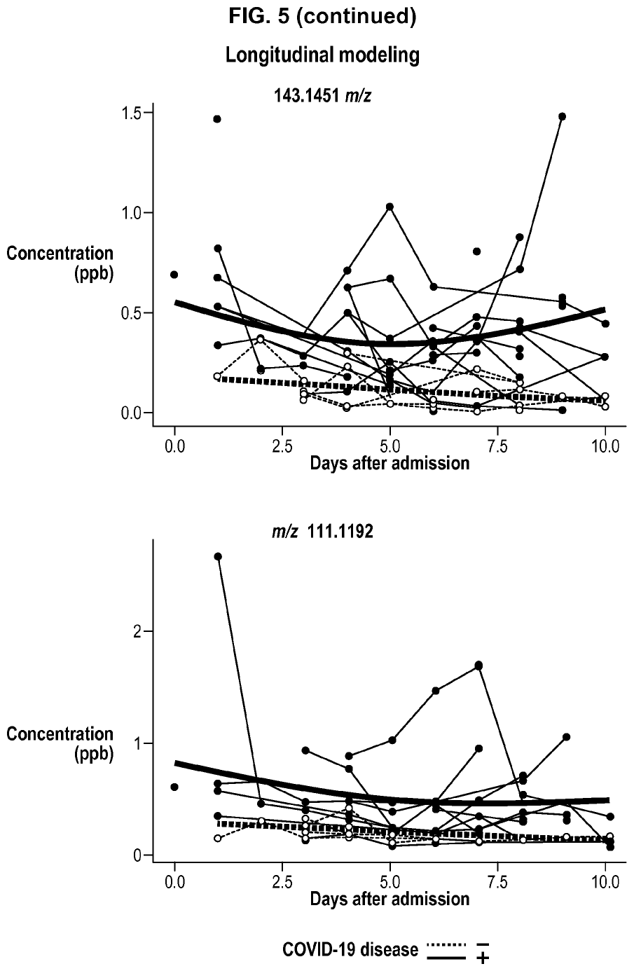

We investigated the expression of the four most prominent VOCs in the whole study population throughout the period of mechanical ventilation (FIG. 5b). FIG. 5 shows the longitudinal analysis of VOCs in exhaled breath. The four features (m/z 99.08, 135.09, 143.15 and 111.12) contributing the most to the models were assessed in the first sample available for each patient (a) and over time (b) during the ICU stay of intubated, mechanically ventilated patients with COVID-19 (in red) or non-COVID-19 ARDS (in blue). All the points for a given patient are connected, and the bold lines correspond to the fixed effect of the mixed model for each group.

We observed that the VOC concentrations (i) were significantly higher in the breath of patients with COVID-19 ARDS than in the breath of patients with non-COVID-19 ARDS, and (ii) tended to decrease over the first 10 days of hospitalization. The putative annotations for the four compounds at m/z 135.09, 143.15, 99.08 and 111.12 were respectively 1-chloroheptane, nonanal, methylpent-2-enal, and 2,4-octadiene.

### Correlation With Viral Load and Severity Scores

The viral load in bronchoalveolar fluid was measured for 18 patients. The median [IQR] value in the first sample was 7.2 [6.2-8.4] log eq. copies/mL. The VOC concentrations in the first sample were not significantly correlated with the bronchoalveolar fluid viral load or with the severity of illness (i.e. the SAPS II and SOFA score^{27,28}) measured during the first 24 hours in the ICU (Table 2).

TABLE 2
Absence of significant correlations between VOC concentrations
and the SAPS II, SOFA score and viral load.

| | SAPS II score | | SOFA score | | Viral load | |
|---|---|---|---|---|---|---|
| VOC (m/z) | r | p-value | r | p-value | r | p-value |
| 143.15 | 0.12 | 0.62 | 0.27 | 0.25 | −0.23 | 0.24 |
| 135.09 | 0.05 | 0.85 | 0.35 | 0.14 | −0.0004 | 1.00 |
| 99.08 | 0.04 | 0.88 | 0.36 | 0.13 | 0.08 | 0.70 |
| 111.12 | 0.02 | 0.93 | 0.28 | 0.25 | −0.14 | 0.48 |

r: Pearson's correlation coefficient.

This study provided proof of concept for the measurement of VOCs and the determination of a specific VOC breathprint in the exhaled breath from patients with COVID-19-related ARDS requiring invasive mechanical ventilation in the ICU. This breathprint was independent of the severity of illness and the viral load. Four features (m/z 99.08, 111.12, 135.09 and 143.15) discriminate between COVID-19 and non-COVID-19 ARDS in the longitudinal analysis.

Two of these four prominent VOCs (m/z=143.15 and m/z=99.08, which may correspond to methylpent-2-enal and nonanal) are aldehydes, while 2,4-octadiene (m/z=111.12) is an alkadiene; all three compounds are known to be expressed in breath.^{38,39} Nonanal (m/z=99.08) is a sub-product of the destruction of the cell membrane as a result of oxidative stress; reactive oxygen species may be generated by various type of inflammatory, immune and structural cell in the airways.⁴⁰ In studies of exhaled breath from patients with ARDS, Schubert et al. found abnormally low isoprene concentrations and Bos et al. reported abnormally high concentrations of octane, acetaldehyde and 3-methylheptane.^{21,22} Differences in study populations (non-COVID-19 vs. COVID-19 ARDS) and analytical methods (offline vs. online) might explain the differences between the VOCs identified in the present study and those identified in previous studies of ARDS.^{21,22}

In line with previous reports, the VOC concentrations measured here were not correlated with the severity of illness (as judged by the SAPS II and the SOFA score).²¹ This finding suggests that the exhaled breath signature is a marker of COVID-19 per se, rather than of the severity of illness. Likewise, the VOC concentrations were not correlated with viral load, suggesting that this signature may be a marker of the disease related to SARS-CoV-2 rather than of virus carriage.

Our interpretation of the present data may have been limited by differences between the COVID-19 and non-COVID-19 ARDS subgroups. Patients with COVID-19 ARDS cohort had higher respiratory rate, FiO₂, PEEP and CRP values on admission. The respiratory rate, PEEP and CRP were not found to be confounding factors, and all the patients were sampled when breathing 100% FiO₂ (to avoid mass spectrometry interference by oxygen).²⁹

The FiO₂ represents the percentage of oxygen in the air with which patients are ventilated, the values range from 21% (ambient air) to 100% (pure oxygen, for the most severe patients). This percentage of oxygen causes interference for analysis with a PTR-MS. For this reason, for all patients, we first performed a first analysis with their basal FiO₂ level (between 21 and 100%), then all patients were ventilated with 100% FiO₂ for 5 mins to have a standardized value and a second analysis was performed. Only the results of this second analysis are detailed here.

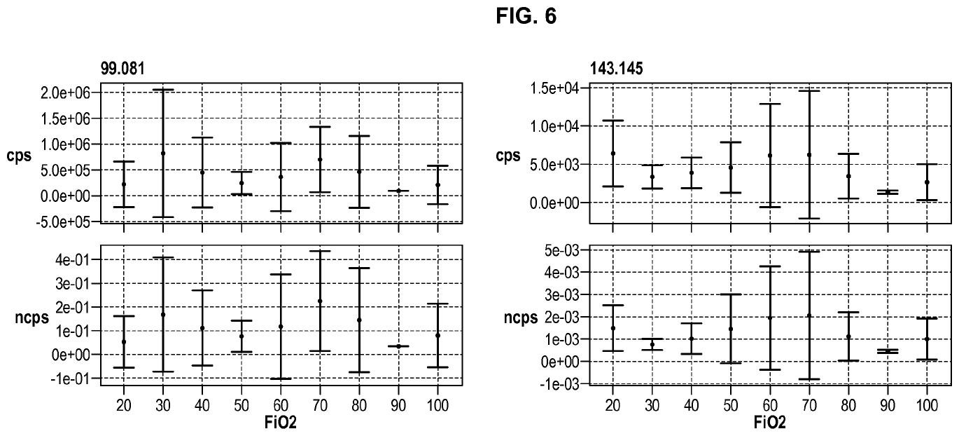

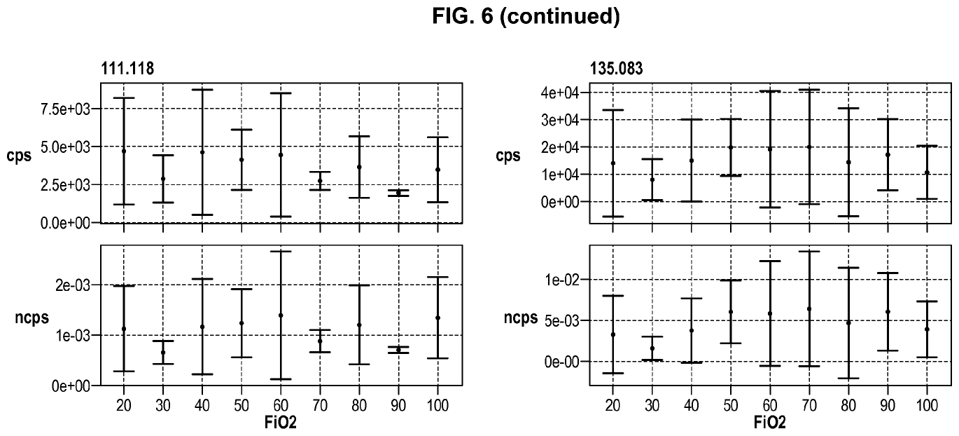

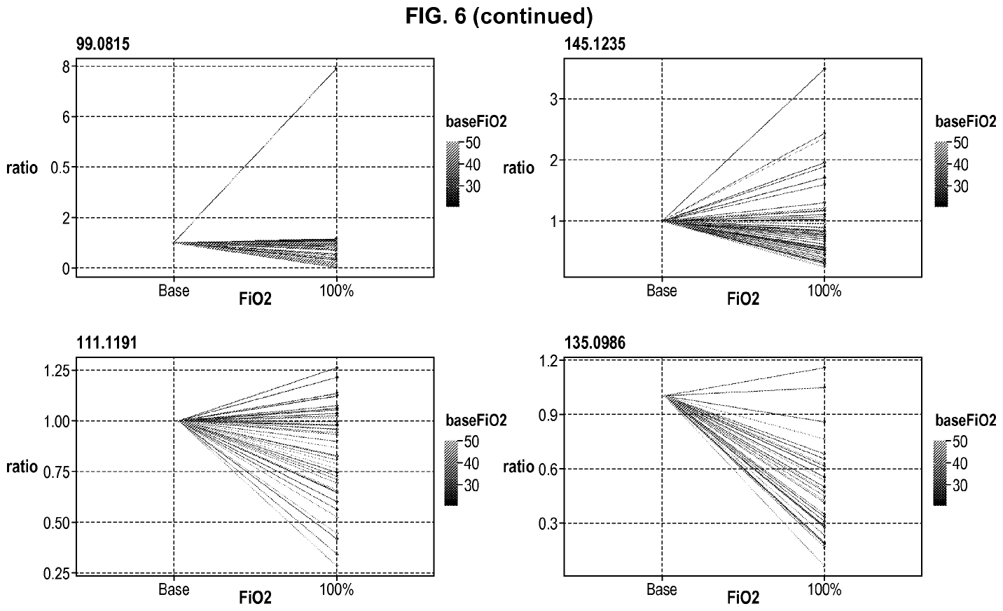

The graphs of FIG. 6 represent, for each of the four compounds of interest, the values measured in the two analyses. The first four graphs show the values obtained for all patients as a function of the initial % FiO₂ (“raw” values (cps, upper line), or normalized (ncps, lower line, normalization process not detailed here). The four other graphs of the figure represent the variations observed for each patient between the 1st measurement (value on the left, “base”) and the 2nd measurement in 100% FiO₂ (value on the right). The statistical analyses performed do not show any significant difference, which suggests that the percentage of oxygen does not disturb the analysis of the 4 compounds of interest.

Similarly, patients with COVID-19 ARDS were more likely to have been treated with hydroxychloroquine and fludrocortisone. However, these drugs were administered to the patients after their first sample had been analyzed. Although the VOC concentrations decreased over time, the treatments did not change. Lastly, there is no correspondence between VOCs described in the present study and the molecular masses of the known metabolites of hydroxychloroquine or fludrocortisone.

The ptairMS R package used for data analysis is fully available at https://github.com/camilleroquencourt/ptairMS.

Due to these findings, we can apply a highly sensitive, rapid, non-invasive, real-time mass spectrometry breath analysis.^{36,37} This contrasts with offline technologies, which require a sampling step and remote, time-consuming analytical steps.^{21,22} The subsequent diagnostic validation and clinical implementation can be based on less cumbersome technologies, such as mass spectrometers dedicated to targeted analyses or portable “electronic noses” with a set of sensors that are relatively selective for different families of VOCs (as previously used in patients with ARDS).²⁰

### EMBODIMENT

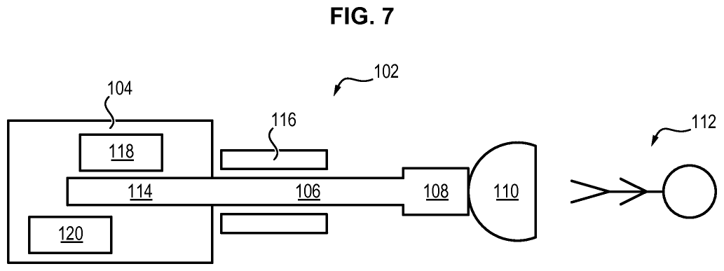

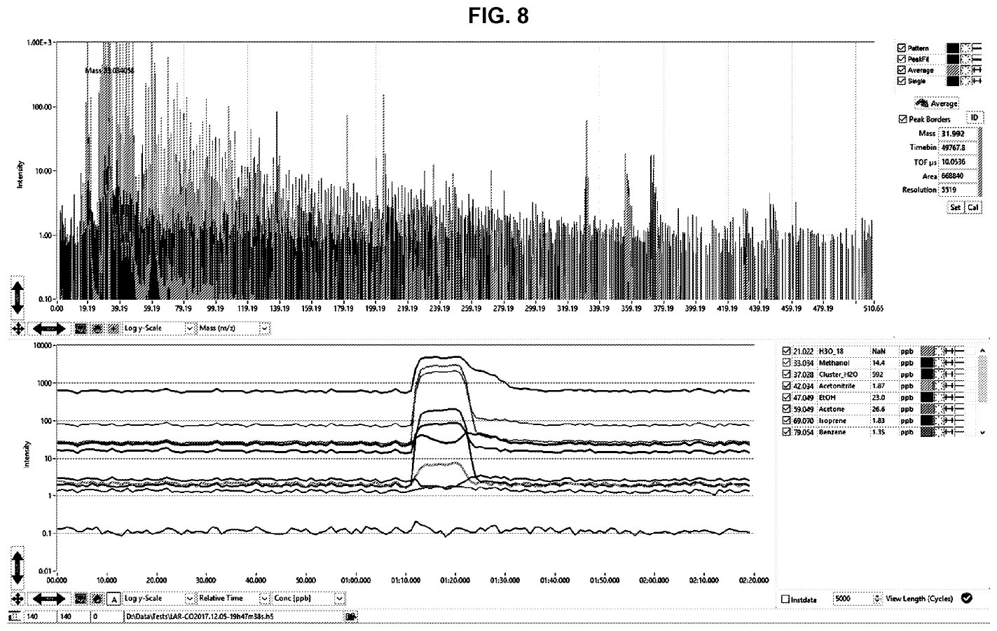

We will now present a first embodiment of the device for screening, diagnosis or monitoring of COVID-19, with reference to FIGS. 7 and 8.

The device 102 comprises a body 104.

The device comprises means for receiving a breath sample. These means comprise an elongated transfer line 106. One end of the transfer line has a connector 108 configured for connecting to an end of an endotracheal tube 110. This tube is configured for installation on the face of a person 112 for invasive mechanical ventilation. Another end 114 of the transfer line 106 extends inside body 104.

The device also comprises means 116 for heating the transfer line, which helps to preserve the components or compounds from the moment they exit the patient to the moment the breath sample is analyzed inside device 102.

The device comprises inside body 104 means configured for determining whether m/z=135.09, m/z=143.15, m/z=99.08 and m/z=111.12 are present in a breath sample. These means comprise in this example a proton-transfer-reaction mass spectrometer 118 configured for analyzing a breath sample transferred through line 106.

The device has computer and/or electronic control means 120. These means comprise a program comprising code instructions configured for controlling device 102 for performing the method presented hereafter. This program can be provided on a communication network in view of downloading the program into the device when it is connected to this network with classical means in this view.

The method of the invention is performed as follows with this device 102.

We assume that the person 112 is undergoing invasive mechanical ventilation. An endotracheal tube 110 is installed on the person. The connector 108 of transfer line 106 is connected to the endotracheal tube 110. The person is breathing for example air of 100% FiO₂, which means that this person inspires pure oxygen.

The method comprises the step of heating the transfer line with the heating means 116.

It then comprises the step of obtaining a sample of exhaled breath from the person 112 via the heated transfer line. Thus, the sample of exhaled breath enters body 104 for being exposed to the spectrometer and analyzed.

The method then comprises the step of determining whether m/z=135.09, m/z=143.15, m/z=99.08 and m/z=111.12 are present in the sample.

To this end, the spectrometer 118 and the control means 120 perform the steps of determining values of signal intensities and/or concentrations of ions defined by their mass-to-charge ratios (m/z) given by the spectrometer in a group of ranges, each range being defined by a median value and limits of the range. In this example, the group consists of the following ranges:
m/z=135.09 (135.03-135.17),
m/z=143.15 (143.07-143.22),
m/z=99.08 (99.04-99.14),
m/z=111.12 (111.07-111.18).

FIG. 8 shows on the second graph the amount of certain components into the breath received inside the device as a function of time. The sample is received inside the device roughly between 1 min 10 s and 1 min 20 s. At that moment, the amount of some components rises and then decreases.

The first graph shows the intensity or concentration of different components as a function of their mass (m/z). Each peak thus designates a different component. The higher the peak, the higher the intensity or concentration of the component in the exhaled breath. The device can directly and in real time evaluate whether one, two, three or all of the eight components are present in the exhaled breath.

For this purpose, the device determines the value m/z of each of the components ([M+H]⁺) in the sample.

Then we use these signal intensities or concentrations to predict the probability of COVID infection thanks to a machine learning model trained with the data. For that, the method comprises the step of applying at least one test or decision rule to the values, the group and the test or decision rule being configured for identifying a patient with COVID-19.

In this embodiment, the tests or decision rules of elastic net, random forest and support vector machine (SVM) can be used for example. This testing step is performed as follows for example.

We log-transform the measured concentration in ppb of k VOCs of interest. We note x=(x₁, . . . , xₖ) those values.

### Elastic Net³²

We generally define the probability that this individual is in the COVID-19 positive group as follows:

$$p = {logit}^{- 1} ( {α}_{0} + {\sum }_{i = 1}^{k} {α}_{i} \times {x}_{i} )$$

where (α₀, . . . , αₖ) are known coefficient estimated from our data, and

$${logit}^{- 1} ( x ) = \frac{exp ( x )}{1 + exp ( x )}$$

If p is greater than a certain threshold (e.g. 0.5), the individual is considered to be in the COVID-19 positive group.

In our example with the four VOC of interest, we note x₉₉, x₁₁₁, x₁₃₅, x₁₄₃ the logarithm of ions 99.08, 111.12, 135.09 and 143.15 concentrations.

After training and tuning the model, we obtain the decision rules:

p=logit⁻¹(4.32692+0.0717×x₉₉−0.88062×x₁₁₁+1.51188×x₁₃₅+1.82797×x₁₄₃)

If p is greater than 0.5, the individual is considered to be in the COVID-19 positive group.

### Random Forest

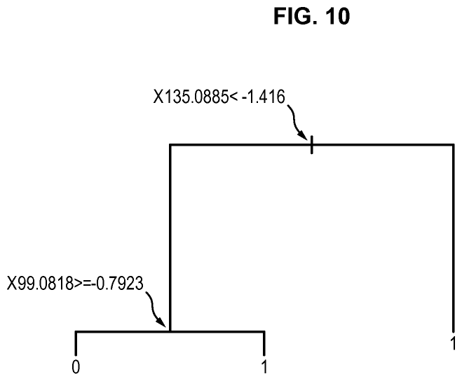

The Random Forest approach is based on the aggregation of the predictions from many decision trees³³. Here, five hundred decision trees as the one illustrated on FIG. 10 have been built from our data. Each tree is built from a random subset of our variables. For the illustrated tree, if the logarithm of the ions 135.09 is less than −1.416, the patient is considered to belong to the positive group. Else, we look at the log-concentration of the ion 99.09. If this is less than −0.7923, the patient is in the negative group. We note the result for the five hundred trees, which are negative (0) or positive (1). If there is more positive outputs than negative outputs, the individual is considered to be in the COVID-19 positive group.

### SVM³⁴

An equation of a hyperplane h(x)=0 separating the two classes has been estimated from our data, for example in the linear case:

$$h ( x ) = {\sum }_{i}^{k} {α}_{k} {x}_{k} + {α}_{0}$$

or in general case:

$$h ( x ) = {\sum }_{j}^{p} {α}_{j} K ( {x}^{i} , x ) + {α}_{0}$$

where (x¹, . . . x^{p}) are the features value for patient used to build the hyperplane (called support vector), K a kernel and (αᵢ) the known estimated coefficients.

If h(x)>0 (respectively h(x)≤0), the patient is in the COVID-19 positive (respectively negative) group.

In our examples with the four VOC of interest, we note x₉₉, x₁₁₁, x₁₃₅, x₁₄₃ the logarithm of ions m/z=99.08, 111.12, 135.09, 143.15 concentration, whose mean and variance have been scaled according to the values from the training data.

After training and tuning the model, we obtain the decision rule:

h(x₉₉, x₁₁₁, x₁₃₅, x₁₄₃)=0.8776471+1.1364761 x₁₃₅+0.8867700 x₁₄₃+0.3212751 x₉₉−0.1639539 x₁₁₁

If h(x₉₉, x₁₁₁, x₁₃₅, x₁₄₃)>0, the patient is in the COVID-19 positive group.

One or at least two of these tests or decision rules are performed.

Then a message is communicated according to the result of the test(s) or decision rule(s). This comprises for example displaying the result of the test or decision rule on a screen of the device, sending an e-mail to a physicist, etc.

The above-examples for the elastic net and SVM models have been given for the four VOC of interest. Here are the values of coefficients for using the elastic net test with the 65 VOC of interest.

TABLE 3
Coefficients of the elastic net test for 65 VOC of interest

| m/z | coef |
|---|---|
| 26.9994 | 0 |
| 27.0105 | 0 |
| 27.0218 | 0 |
| 27.9951 | 0 |
| 29.0026 | 0 |
| 29.014 | −0.17099834952128 |
| 29.0375 | 0 |
| 30.9838 | −0.19272254930386 |
| 31.0035 | 0 |
| 31.018 | 0 |
| 31.0545 | −0.0868809541462363 |
| 33.0338 | 0 |
| 38.0341 | −0.373544816475641 |
| 39.0231 | 0 |
| 39.033 | 0 |
| 41.0035 | 0 |
| 41.0392 | 0 |
| 43.0553 | 0 |
| 43.9676 | 0 |
| 43.9907 | 0 |
| 44.9981 | 0 |
| 45.0342 | 0 |
| 47.0135 | 0 |
| 47.05 | 0 |
| 51.0446 | 0 |
| 52.9618 | 0 |
| 53.002 | 0 |
| 53.0393 | 0 |
| 55.0549 | −0.204060119119194 |
| 57.0346 | 0 |
| 57.0706 | 0 |
| 60.0531 | 0 |
| 61.0356 | 0 |
| 62.0299 | −0.172910672454371 |
| 63.0079 | −0.133793047603517 |
| 65.0606 | 0 |
| 66.0475 | 0 |
| 66.9591 | 0 |
| 67.055 | −0.0185542639450894 |
| 68.0625 | 0 |
| 69.0706 | 0 |
| 71.0501 | −0.29614511174346 |
| 71.0861 | 0 |
| 73.0656 | 0 |
| 75.045 | 0 |
| 77.0604 | 0 |
| 79.0553 | 0.1565639655472 |
| 81.019 | 0 |
| 81.0708 | 0 |
| 83.0866 | −0.264806002067097 |
| 85.1024 | 0 |
| 87.0815 | 0 |
| 89.061 | 0 |
| 91.0563 | 0 |
| 93.0378 | 0.0134534777254841 |
| 97.0667 | 0.142381904312499 |
| 99.0818 | 0.116216917765426 |
| 107.0865 | 0.0435199756862549 |
| 111.1192 | 0.139762358652873 |
| 115.1135 | 0 |
| 117.0928 | 0 |
| 135.0885 | 0.34647275629029 |
| 137.1346 | 0.0746880721623011 |
| 143.1451 | 0.548099225194985 |
| 179.1459 | 0 |

Here are the values of coefficients for using the SVM test with the 65 VOC of interest.

TABLE 4
Coefficients of the SVM test for 65 VOC of interest

| features | coef |
|---|---|
| intercept | 1.78457393935063 |
| 26.9994 | 0.0203164775583294 |
| 27.0105 | −0.00646293739846489 |
| 27.0218 | 0.0131373528739178 |
| 27.9951 | 0.00396558638862555 |
| 29.0026 | 0.0105578163957263 |
| 29.014 | −0.0490358531227796 |
| 29.0375 | 0.0122541721514019 |
| 30.9838 | −0.0794211818615996 |
| 31.0035 | −0.0371246281462103 |
| 31.018 | −0.00301229548201719 |
| 31.0545 | −0.099084453254533 |
| 33.0338 | −0.00907141264166196 |
| 38.0341 | −0.029855704498521 |
| 39.0231 | −0.0397990392900494 |
| 39.033 | 0.00637568953724734 |
| 41.0035 | −0.0230482514619407 |
| 41.0392 | 0.011438473256849 |
| 43.0553 | 0.0526010770568651 |
| 43.9676 | −0.00876987827383436 |
| 43.9907 | 0.0229029819998173 |
| 44.9981 | 0.00858726062702962 |
| 45.0342 | −0.00751150864277307 |
| 47.0135 | 0.0140843908608015 |
| 47.05 | −0.0152800023536971 |
| 51.0446 | −0.0276965017027041 |
| 52.9618 | −0.0294181148271273 |
| 53.002 | −0.0114321026066049 |
| 53.0393 | −0.0329220742342297 |
| 55.0549 | −0.0796086805043102 |
| 57.0346 | −0.0156023981925246 |
| 57.0706 | 0.0165741125645432 |
| 60.0531 | 0.0169707074015356 |
| 61.0356 | 0.0476857892192711 |
| 62.0299 | 0.00128519033348025 |
| 63.0079 | −0.0347890132637384 |
| 65.0606 | −0.00565899268637548 |
| 66.0475 | −0.0159928201806251 |
| 66.9591 | −0.00725756349009642 |
| 67.055 | −0.0442809159240086 |
| 68.0625 | −0.0224115129980686 |
| 69.0706 | −0.0183969983383 |
| 71.0501 | −0.074340950426599 |
| 71.0861 | 0.0085464014404322 |
| 73.0656 | −0.0358032773974026 |
| 75.045 | 0.00282342778864358 |
| 77.0604 | −0.0216485052925101 |
| 79.0553 | 0.0323336124905708 |
| 81.019 | −0.0225294351687507 |
| 81.0708 | 0.0121522697786908 |
| 83.0866 | −0.0769266668049326 |
| 85.1024 | 0.012202175586782 |
| 87.0815 | −0.00812314515499904 |
| 89.061 | 0.0196373308932 |
| 91.0563 | 0.00999750102871999 |
| 93.0378 | 0.040514992832867 |
| 97.0667 | 0.0308882660672048 |
| 99.0818 | 0.080845663301971 |
| 107.0865 | 0.0264354320284494 |
| 111.1192 | 0.0274064508385647 |
| 115.1135 | 0.0518974143614893 |
| 117.0928 | 0.0379433650316464 |
| 135.0885 | 0.0901668878172821 |
| 137.1346 | 0.028631519713326 |
| 143.1451 | 0.0981198699268599 |
| 179.1459 | −0.0135128151592857 |

Thus, this method uses real-time, online, proton transfer reaction time-of-flight mass spectrometry to perform a metabolomic analysis of exhaled breath from adults undergoing invasive mechanical ventilation in an intensive care unit due to severe COVID-19 or non-COVID-19 acute respiratory distress syndrome (ARDS). It is a real-time, non-invasive detection in exhaled breath for identifying patients with COVID-19.

### Second Example

We now present PTR-MS breath analysis results for SARS-CoV-2 in and spontaneously breathing persons (non-intubated) who may carry or be infected with SARS-CoV-2, made with the invention.

These results were obtained thanks to the VOC-COVID-DIAG (NCT04614883) and VOC-SARSCOV-DEP (NCT04817371) clinical studies promoted by the Foch

Hospital and for which the scientific coordinators and principal investigators are Pr Philippe Devillier and Dr Hélène Salvator.

The aim of these studies was to validate the value of volatile organic compounds (VOCs) analysed by mass spectrometry, electronic noses and/or olfaction for the diagnosis of SARS-CoV-2 infection in patients admitted to hospital with suspected SARS-CoV-2 (COVID-19) or other respiratory-tropic viruses, or hospitalised with systematic screening for SARS-CoV-2 infection, as well as in vaccinated volunteers. For each patient, a breath sample was taken for analysis and a sweat sample for VOC analysis by canine olfaction or other non-detailed mass spectrometry techniques. A symptom score was also established by the investigator, on a scale of 0 to 4, according to the presence of certain symptoms (fever, fatigue, cough, diarrhoea, anosmia, agueusia, headache, dyspnoea, polypnoea, hypoxia . . . ).

The results concerning the invention relate to PTR-MS analysis and are an analysis of the data available for a total of 136 patients that can be used with this technology.

While the results of the first example involved intubated and mechanically ventilated patients in an intensive care unit, the patients in these studies were spontaneously breathing and therefore the experimental design had some differences. Although we will designate them collectively for simplicity as “patients” in the following description of this example, some of them where not actually suffering from COVID-19 and where merely persons possibly carrying or infected by SARS-CoV-2, with or without clinical symptoms.

Here, the patients' exhaled breath was collected on the ward in a special exhaled breath collection bag (Ted lar bag®) by exhaling the patient into the bag using the appropriate device. The bag, once closed, was then transported to the analysis room. A flexible tube was then connected to the Tedlar bag and allowed the contents of the bag to be aspirated directly into the mass spectrometer for real-time analysis at a flow rate of 50 ml/min. Other analytical and data processing procedures were similar to those described above.

The patients were divided into three groups:
no infection: SARS-CoV-2 PCR negative: 69 patients (50 vaccinated and 19 unvaccinated);
old infection: SARS-CoV-2 PCR negative but history of COVID-19 infection in the previous weeks or months: 12 patients; and
recent infection: SARS-CoV-2 PCR positive: 55 patients.

The data were analysed with the ptairMS software developed by Camille Roquencourt and Etienne Thévenot (CEA).

### 1—Univariate Analysis

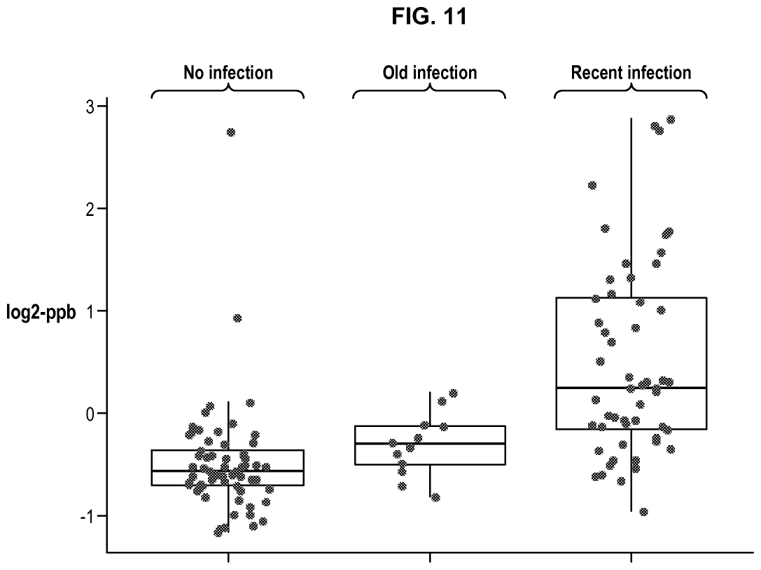

Univariate analysis showed a significant difference between groups for the m/z 99.08 signal (p-value<0.001 Wilcoxon test), which was one of the 4 significant signals in the study in intensive care patients. The graph representing the level of expression of the m/z 99.08 signal according to the groups of patients is presented on FIG. 11.

The following points should be noted.

In the “Recent infection” group, illustrated on the right, the 12 patients with the lowest signal intensity had specificities, in particular 5 of them had received prior treatment with dexamethasone (anti-inflammatory glucocorticoid drug) between 1 and 9 days before sampling; 2 were organ transplant patients (kidney or lung) and under immunosuppressive treatment, 1 patient was asthmatic with inhaled glucocorticoid treatment.

The “Old infection” group is illustrated at the center.

In the “No infection” group, illustrated on the left, the two patients with the highest intensities were patients with other infections, namely clostridial colitis and septicaemia (Escherichia coli positive blood cultures).

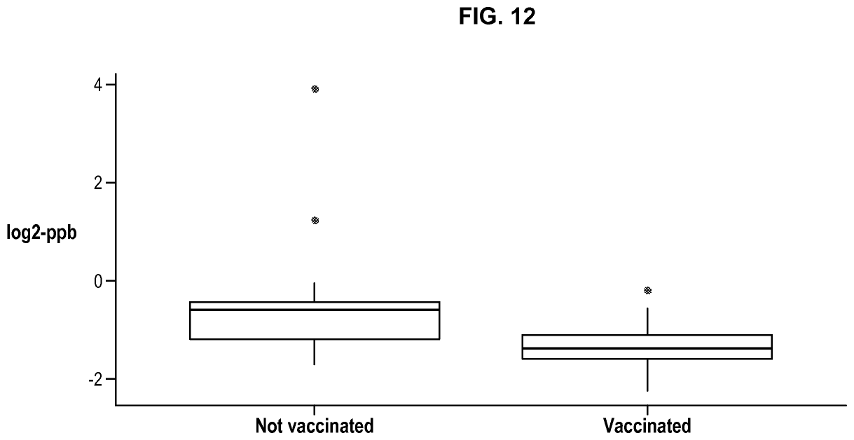

In the “No infection” group, 50 patients were vaccinated and 19 non-vaccinated. No significant difference in m/z 99.08 signal intensity was observed between vaccinated and non-vaccinated patients (see FIG. 12), suggesting that vaccination status does not interfere with the proposed screening method.

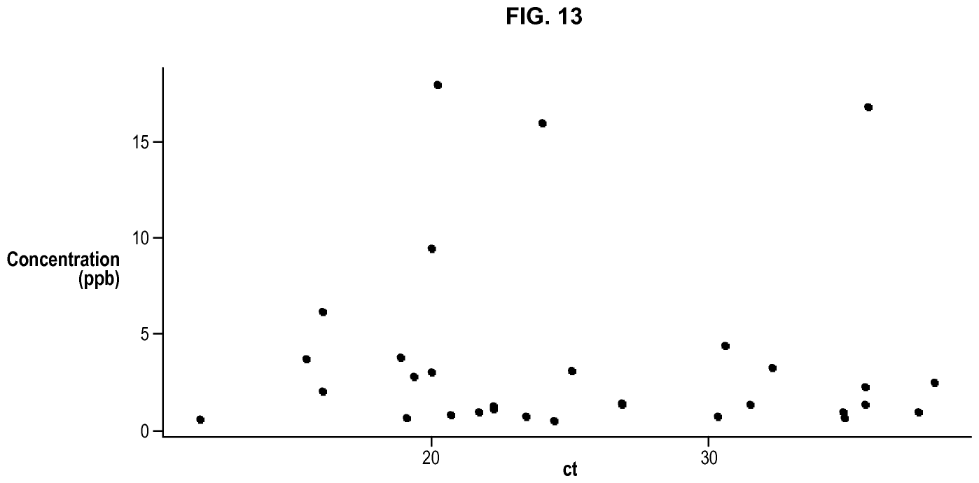

We then assessed whether the m/z 99.08 signal intensity correlated with the Ct (cycle threshold) value measured by the PCR assays on nasopharyngeal swabs when this value was available in the ‘Recent Infection’ group. This is obtained for SARS-CoV-2 detection with reverse-transcriptase quantitative polymerase chain reaction (RT-qPCR) on nasopharyngeal swabs. Ct is a reflection of the viral load present in each sample: the higher the number of copies of the SARS-CoV-2 gene mRNA detected by the test, the lower the Ct. The results are presented on FIG. 13 and show the lack of correlation between the two parameters, suggesting that breath testing is likely to detect patients with high and low viral loads in the same way.

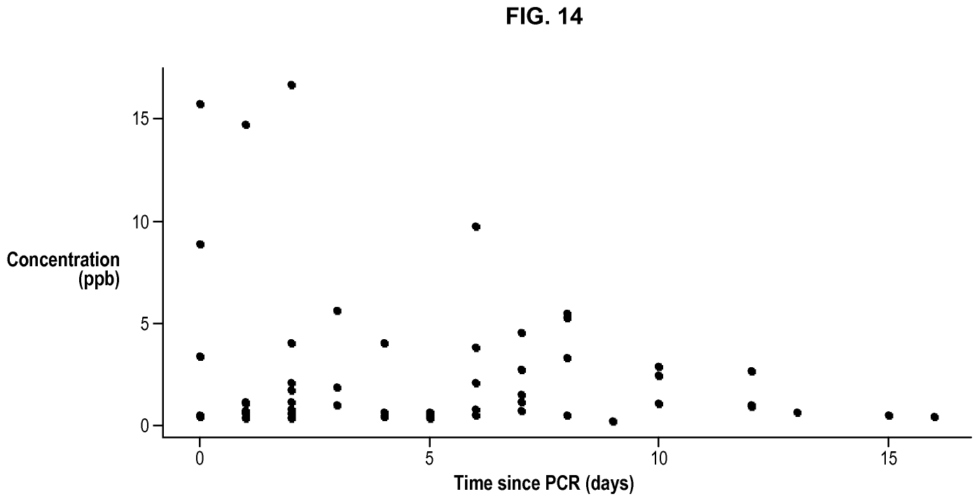

In the same group, we also looked to see if the m/z 99.08 signal intensity correlated with the time elapsed between the PCR being performed. The results are presented on FIG. 14 and show that there was no correlation and suggest that breath testing can detect very recent infections in the same way as less recent infections (maximum delay of 15 days).

This example shows that we may perform a method for identifying that a person carries or is infected by SARS-CoV-2 or that a person does not carry or is not infected by SARS-CoV-2, the method comprising:
obtaining a sample comprising elements coming from breath exhaled from a person;
exposing the sample to a spectrometer;
determining only one value of signal intensity and/or concentration of ions defined by its mass-to-charge ratio (m/z) given by the spectrometer in only one range comprising m/z=99.08 (99.04-99.14),
applying at least one test to the value, and
communicating a message according to a result of the test.

The test comprises for example the step of determining whether the intensity or concentration is above a predetermined threshold, for example 1.67 ppb (hence the person carries or is infected with SARS-CoV-2) or not (hence the person does not carry or is not infected with SARS-CoV-2). With a logistic regression based on the concentration of the m/z 99.08, a threshold estimated at 1.67 ppb allows the detection of the carriage or infection with SARS-CoV-2 with an accuracy of 74% (40% precision, 97% recall and AUC 0.853).

### 2—Multivariate Analysis

### A—Untargeted Analysis

After the steps of alignment, imputation, isotope suppression, saturated ions (m/z 37 and m/z 59) and oxygen ions, and outlier suppression, we obtain a matrix of 82 VOCs and 134 patients. For this analysis, only two groups were considered: patients with negative PCR test for SARS-CoV-2 (corresponding to the two “No infection” and “Old infection” groups of the univariate analysis) and patients with positive PCR test for SARS-CoV-2, resulting in 55 positive patients and 79 negative patients.

Five data were added to build the model:
previous corticosteroid therapy before sampling (0 no, 1 yes),
vaccinal status for COVID-19 (0 no, 1 yes),
symptom score (0 to 4),
oxygenotherapy during the sampling (0 no, 1 yes), and
   time since PCR in days.

We then used the Elastic Net model to predict the COVID-19 status. To select the best penalization parameters, we performed a 10-fold cross validation, repeated 4 times, with a proportion of 40% infected and 60% not infected patients in each fold. Parameters giving the best accuracy were selected.

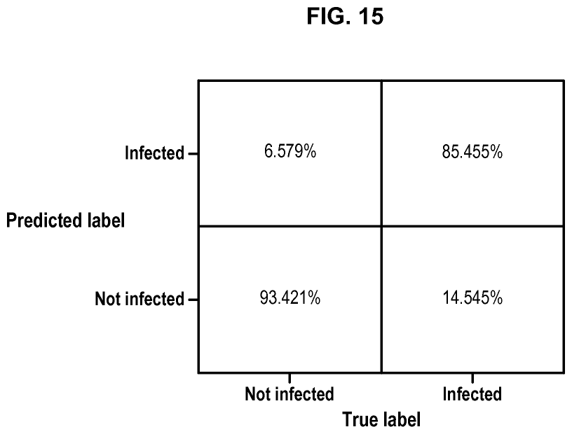

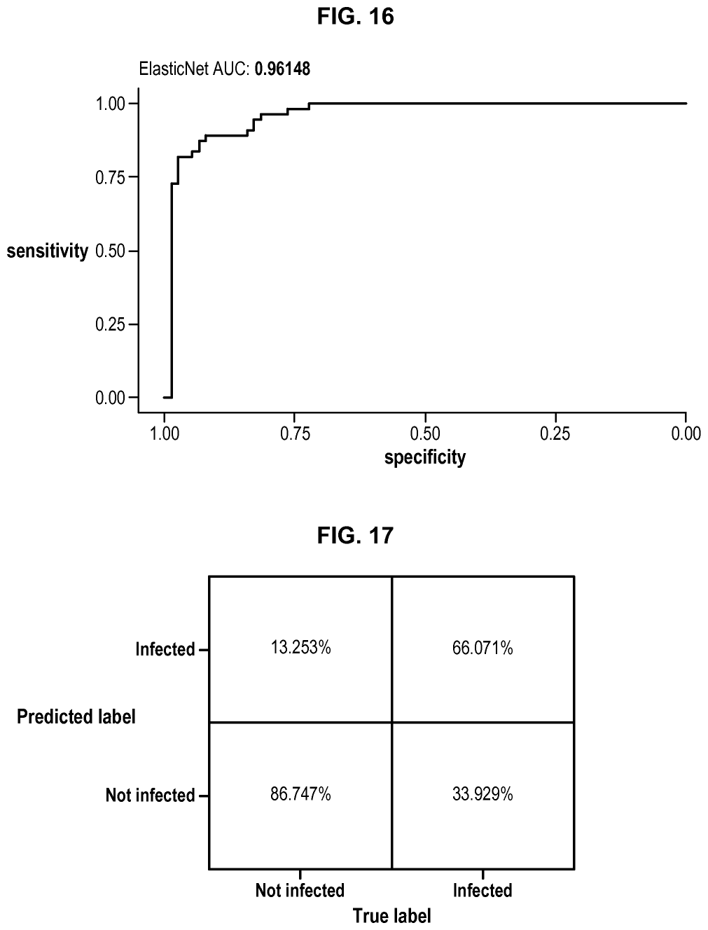

The selected model, which includes the signal intensity of feature m/z 99.08, the symptom score, previous corticosteroid therapy before sampling and oxygenatherapy, allows the detection of COVID-19 with an accuracy of 91% in cross validation. The diagnostic performances are presented in the graph of FIG. 16, with an area under the Receiver operating characteristic curve (AUC) of 0.961, which it close to 1. FIG. 15 shows the confusion matrix with a precision of 85.5% and recall 93.4%.

Thus, this example also shows that we may perform a method for identifying that a person carries or is infected by SARS-CoV-2 or that a person does not carry or is not infected by SARS-CoV-2, the method comprising:
obtaining a sample comprising elements coming from breath exhaled from a person;
exposing the sample to a spectrometer;
determining only one value of signal intensity and/or concentration of ions defined by its mass-to-charge ratio (m/z) given by the spectrometer in only one range comprising m/z=99.08 (99.04-99.14),
determine the symptom score, whether the person had a corticosteroid and/or oxygen therapy before sampling, and
applying at least one test to the value to predict the probability of the patient carrying or being infected by SARS-CoV-2 (as described for the model elastic Net)

### B—Targeted Analysis

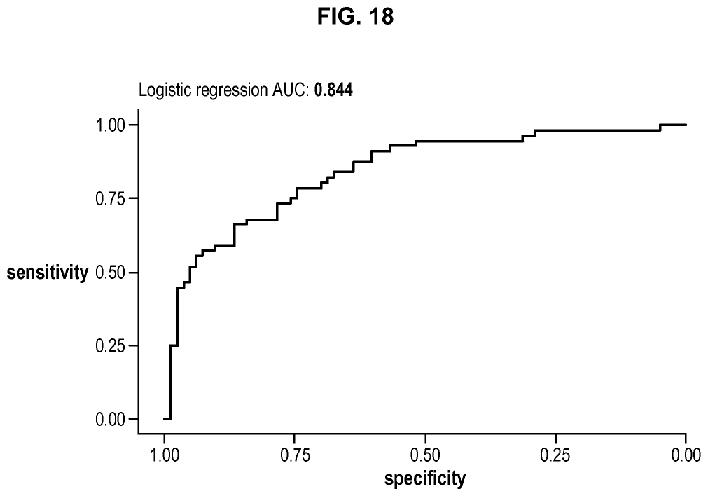

In this second example, a complementary analysis was made using the same model as the model used for intubated, mechanically ventilated patients, using only the concentration of the four signals m/z 99.08, m/z 111.12, m/z 135.10 and m/z 143.14. It shows that we predict the COVID-19 status with an accuracy of 78% in 10-fold cross validation, with a logistic regression. The confusion matrix of the model and the Receiver operating characteristic curve are presented on FIGS. 17 and 18.

In this case, the example shows that we may perform a method for identifying that a person carries or is infected by SARS-CoV-2 or not, the method comprising:
obtaining a sample comprising elements coming from breath exhaled from a person;
exposing the sample to a spectrometer;
determining values of signal intensities and/or concentrations of ions defined by their mass-to-charge ratios (m/z) given by the spectrometer in a group comprising the following ranges:
   m/z=135.09 (135.03-135.17),
   m/z=143.15 (143.07-143.22),
   m/z=99.08 (99.04-99.14), and
   m/z=111.12 (111.07-111.18).
applying at least one test to the values, and
communicating a message according to a result of the test.

Apart from that, the method is performed in the same way as in the first example.

### Conclusion

The invention is not limited to the above-described embodiments and many modifications of these embodiments could be made without departing from the invention.

Other types of spectrometer may be used. But this kind of spectrometer can directly receive exhaled breath and provide immediately the result of the analysis. Other kinds of spectrometer may need more operations and deliver the result after a certain period of time.

For a given M molecule, the m/z values indicated above are characteristic of the mass spectrometer we used (PTR-TOF-MS). With this type of instrument, the molecule M whose mass would be equal to 200 is detected in its form [M+H]⁺ (the spectrometer “grafts” a proton to the molecule in order to give it an electric charge making detection possible), i.e. m/z=201. It would also be possible to detect the same molecule M with other types of mass spectrometers based on different technologies, for example GC-MS, and in this case for this same molecule M, we would obtain masses corresponding to 4 or 5 fragments, for example 72, 125, 140 and 172. Still other types of mass spectrometers could also give values strictly identical to ours (SESI-MS . . . ). Accordingly, the same range may be defined by different median values and limits of the range.

Examples include gas chromatography mass spectrometry (GCMS), liquid chromatography mass spectrometry (LCMS) and proton transfer reaction mass spectrometry (PTR-MS)), ion mobility spectrometry, field asymmetric ion mobility spectrometry, differential mobility spectrometry (DMS); infrared spectroscopy (IR spectroscopy) such as near-infrared (NIR), selected ion flow tube mass spectrometry (SIFT), secondary electrospray ionization (SESI) mass spectrometry, Fourier Transform-Infrared (FOR) spectroscopy and ring-down, cavity spectroscopy or light absorption spectroscopy.

Mass spectrometry can be used in tandem with chromatographic separation techniques. For example. GCMS is an analytical method that combines the features of gas chromatography and mass spectrometry to identify compounds. Similarly, liquid chromatography mass spectrometry (LCMS) is an analytical method that combines the features of liquid chromatography and mass spectrometry to identify compounds.

Ion-mobility spectrometry (IMS) is an analytical technique used to separate and identify ionized molecules in the gas phase based on their mobility in a carrier buffer gas. This technique can be coupled with mass spectrometry and/or chromatographic separation techniques. Having the person inspire pure oxygen is not compulsory. Other percentages of FiO₂ may be used.

This device may be used on a person undergoing invasive mechanical ventilation, but it may also be used on a person who is not undergoing that. It could be a person who is sick or suspected to be sick and/or who has been diagnosed as affected by the COVID-19. It could be a person having ARDS or no ARDS. It could be a person having symptoms or no symptoms. For persons not undergoing mechanical ventilation, sampling devices other than the transfer line directly connected to the end of the endotracheal tube may be used, such as the Buffered End-Tidal Breath Sampling Inlet (BET-med device, lonicon, Innsbruck).

The number of VOC (or ranges of m/z) of interest has been illustrated with examples of 1, 4, 8 and 65. But many other numbers are possible, for example 10, 12, 20, 30, etc., provided a test or decision rule is properly designed to be used with this number of VOC.

As shown, this number of VOC may also be only one. In this case, the VOC could correspond to any of the following ranges already mentioned in the group of 8:
m/z=135.09 (135.03-135.17),
m/z=143.15 (143.07-143.22),
m/z=99.08 (99.04-99.14),
m/z=111.12 (111.07-111.18),
m/z=71.05 (71.02-71.09),
m/z=55.05 (55.03-55.09),
m/z=83.09 (83.05-83.13), and
m/z=93.04 (93.00-93.09).

Besides, when using a number of VOC less than 65, the chosen VOC (or ranges of m/z) of interest need not be among the most prominent ones in the complete signature of 65. A good signature may be obtained with a number of VOC (and an associated test or decision rule) not among the most prominent ones.

1-16. (canceled)

17. A method for analysis, comprising:
exposing a sample comprising elements coming from breath exhaled from a person to a spectrometer;
determining at least one value of a signal intensity and/or concentration of ions defined by a mass-to-charge ratio (m/z) given by the spectrometer in at least one range, the at least one range being defined by a median, a lower limit, and an upper limit;
applying at least one test to the at least one value,
the at least one range and the at least one test being configured for identifying at least one of the following results:
a person carries or is infected by SARS-CoV-2, with or without clinical symptoms,
a person does not carry or is not infected by SARS-CoV-2,
a person suffers from COVID-19, and/or
a person does not suffer from COVID-19; and
communicating the at least one result of the at least one test as a message.

18. The method according to claim 17, wherein the spectrometer is a mass spectrometer.

19. The method according to claim 17, wherein the step of determining comprises determining only one value of signal intensity and/or concentration of ions defined by its mass-to-charge ratio (m/z) given by the spectrometer in only one range.

20. The method according to claim 19, wherein the range comprises m/z=99.08 (99.04-99.14).

21. The method according to claim 17, wherein the step of determining comprises determining values of signal intensities and/or concentrations of ions defined by their mass-to-charge ratios (m/z) given by the spectrometer in a group of ranges.

22. The method according to claim 17, wherein the range is comprised in the following table.

| (m/z [M + H]⁺) | (m/z [M + H]⁺) | (m/z [M + H]⁺) |
|---|---|---|
| median | lower limit | upper limit |
| 135.09 | 135.03 | 135.17 |
| 143.15 | 143.07 | 143.22 |
| 99.08 | 99.04 | 99.14 |
| 111.12 | 111.07 | 111.18 |
| 71.05 | 71.02 | 71.08 |
| 55.05 | 55.03 | 55.09 |
| 83.09 | 83.05 | 83.13 |
| 93.04 | 93.00 | 93.09 |
| 29.01 | 29.00 | 29.03 |
| 115.11 | 115.05 | 115.18 |
| 97.07 | 97.02 | 97.10 |
| 63.01 | 62.98 | 63.04 |
| 47.05 | 47.03 | 47.08 |
| 65.06 | 65.03 | 65.10 |
| 179.15 | 179.07 | 179.25 |
| 73.07 | 73.04 | 73.11 |
| 30.98 | 30.97 | 31.00 |
| 79.06 | 79.02 | 79.10 |
| 81.07 | 81.04 | 81.12 |
| 38.03 | 38.02 | 38.06 |
| 39.02 | 39.01 | 39.04 |
| 53.04 | 53.02 | 53.07 |
| 85.10 | 85.07 | 85.15 |
| 81.02 | 80.98 | 81.06 |
| 41.00 | 40.98 | 41.03 |
| 117.09 | 117.04 | 117.16 |
| 43.99 | 43.97 | 44.02 |
| 31.00 | 30.99 | 31.02 |
| 71.09 | 71.06 | 71.13 |
| 45.03 | 45.01 | 45.06 |
| 43.97 | 43.94 | 43.99 |
| 89.06 | 89.02 | 89.11 |
| 66.96 | 66.93 | 67.00 |
| 107.09 | 107.03 | 107.15 |
| 91.06 | 91.02 | 91.11 |
| 53.00 | 52.97 | 53.03 |
| 75.05 | 75.01 | 75.09 |
| 27.00 | 26.98 | 27.01 |
| 31.02 | 31.00 | 31.03 |
| 67.06 | 67.03 | 67.09 |
| 60.05 | 60.03 | 60.09 |
| 45.00 | 44.98 | 45.02 |
| 39.03 | 39.01 | 39.05 |
| 27.01 | 26.99 | 27.03 |
| 33.03 | 33.02 | 33.05 |
| 41.04 | 41.02 | 41.06 |
| 29.00 | 28.99 | 29.02 |
| 77.06 | 77.03 | 77.10 |
| 31.05 | 31.04 | 31.07 |
| 61.04 | 61.00 | 61.07 |
| 28.00 | 27.98 | 28.01 |
| 57.03 | 57.01 | 57.06 |
| 137.13 | 137.07 | 137.21 |
| 57.07 | 57.05 | 57.10 |
| 62.03 | 62.00 | 62.06 |
| 68.06 | 68.04 | 68.10 |
| 27.02 | 27.01 | 27.04 |
| 43.06 | 43.04 | 43.08 |
| 87.08 | 87.04 | 87.13 |
| 47.01 | 46.99 | 47.04 |
| 52.96 | 52.94 | 52.99 |
| 66.05 | 66.02 | 66.08 |
| 51.04 | 51.02 | 51.07 |
| 69.07 | 69.05 | 69.11 |
| 29.04 | 29.02 | 29.05 |

23. The method according to claim 17, further comprising taking into account at least one of the following elements:
a symptom score,
whether the person had a corticosteroid therapy before sampling, and
whether the person had oxygenotherapy.

24. The method according to claim 17, wherein the person is undergoing invasive mechanical ventilation.

25. The method according to claim 17, wherein the sample is a sample of exhaled breath.

26. The method according to claim 17, comprising obtaining the sample via a transfer line in communication with the person.

27. A device for diagnosis, screening or monitoring of SARS-CoV-2 and/or COVID-19,comprising:
means for determining at least one value of a signal intensity and/or concentration of ions defined by a mass-to-charge ratio (m/z) given by a spectrometer in at least one range,
the at least one range being defined by a median and limits of the range;
means for applying at least one test to the at least one value,
the at least one range and the at least one test being configured for identifying that:
a person carries and/or is infected with SARS-CoV-2,
a person does not carry and/or is not infected with SARS-CoV-2,
a person suffers from COVID-19, and/or
a person does not suffer from COVID-19.

28. The device of claim 27, further comprising a spectrometer.

29. A non-transitory computer-readable storage medium having instructions thereon that, when executed by a processor, cause the processor to execute operation for analyzing a sample according to claim 17.

30. The method according to claim 17, wherein the spectrometer is a proton transfer reaction mass spectrometer.

31. The method according to claim 20, wherein the method comprises identifying that a person is carrying or is infected by SARS-CoV-2 or that a person is not carrying or is not infected by SARS-CoV-2.

32. The method according to claim 21, wherein the group comprises the following ranges:
m/z=135.09 (135.03-135.17),
m/z=143.15 (143.07-143.22),
m/z=99.08 (99.04-99.14), and
m/z=111.12 (111.07-111.18).

33. The method according to claim 21, wherein the group consists in the following ranges:
m/z=135.09 (135.03-135.17),
m/z=143.15 (143.07-143.22),
m/z=99.08 (99.04-99.14), and
m/z=111.12 (111.07-111.18).

34. The method according to claim 21, wherein the group comprises the following ranges:
m/z=135.09 (135.03-135.17),
m/z=143.15 (143.07-143.22),
m/z=99.08 (99.04-99.14),
m/z=111.12 (111.07-111.18),
m/z=71.05 (71.02-71.09),
m/z=55.05 (55.03-55.09),
m/z=83.09 (83.05-83.13), and
m/z=93.04 (93.00-93.09).

35. The method according to claim 21, wherein the group consists in the following ranges:
m/z=135.09 (135.03-135.17),
m/z=143.15 (143.07-143.22),
m/z=99.08 (99.04-99.14),
m/z=111.12 (111.07-111.18),
m/z=71.05 (71.02-71.09),
m/z=55.05 (55.03-55.09),
m/z=83.09 (83.05-83.13), and
m/z=93.04 (93.00-93.09).

36. The method according to claim 21, wherein the group comprises at least one of the ranges of the following table:

| (m/z [M + H]⁺) | (m/z [M + H]⁺) | (m/z [M + H]⁺) |
|---|---|---|
| median | lower limit | upper limit |
| 135.09 | 135.03 | 135.17 |
| 143.15 | 143.07 | 143.22 |
| 99.08 | 99.04 | 99.14 |
| 111.12 | 111.07 | 111.18 |
| 71.05 | 71.02 | 71.08 |
| 55.05 | 55.03 | 55.09 |
| 83.09 | 83.05 | 83.13 |
| 93.04 | 93.00 | 93.09 |
| 29.01 | 29.00 | 29.03 |
| 115.11 | 115.05 | 115.18 |
| 97.07 | 97.02 | 97.10 |
| 63.01 | 62.98 | 63.04 |
| 47.05 | 47.03 | 47.08 |
| 65.06 | 65.03 | 65.10 |
| 179.15 | 179.07 | 179.25 |
| 73.07 | 73.04 | 73.11 |
| 30.98 | 30.97 | 31.00 |
| 79.06 | 79.02 | 79.10 |
| 81.07 | 81.04 | 81.12 |
| 38.03 | 38.02 | 38.06 |
| 39.02 | 39.01 | 39.04 |
| 53.04 | 53.02 | 53.07 |
| 85.10 | 85.07 | 85.15 |
| 81.02 | 80.98 | 81.06 |
| 41.00 | 40.98 | 41.03 |
| 117.09 | 117.04 | 117.16 |
| 43.99 | 43.97 | 44.02 |
| 31.00 | 30.99 | 31.02 |
| 71.09 | 71.06 | 71.13 |
| 45.03 | 45.01 | 45.06 |
| 43.97 | 43.94 | 43.99 |
| 89.06 | 89.02 | 89.11 |
| 66.96 | 66.93 | 67.00 |
| 107.09 | 107.03 | 107.15 |
| 91.06 | 91.02 | 91.11 |
| 53.00 | 52.97 | 53.03 |
| 75.05 | 75.01 | 75.09 |
| 27.00 | 26.98 | 27.01 |
| 31.02 | 31.00 | 31.03 |
| 67.06 | 67.03 | 67.09 |
| 60.05 | 60.03 | 60.09 |
| 45.00 | 44.98 | 45.02 |
| 39.03 | 39.01 | 39.05 |
| 27.01 | 26.99 | 27.03 |
| 33.03 | 33.02 | 33.05 |
| 41.04 | 41.02 | 41.06 |
| 29.00 | 28.99 | 29.02 |
| 77.06 | 77.03 | 77.10 |
| 31.05 | 31.04 | 31.07 |
| 61.04 | 61.00 | 61.07 |
| 28.00 | 27.98 | 28.01 |
| 57.03 | 57.01 | 57.06 |
| 137.13 | 137.07 | 137.21 |
| 57.07 | 57.05 | 57.10 |
| 62.03 | 62.00 | 62.06 |
| 68.06 | 68.04 | 68.10 |
| 27.02 | 27.01 | 27.04 |
| 43.06 | 43.04 | 43.08 |
| 87.08 | 87.04 | 87.13 |
| 47.01 | 46.99 | 47.04 |
| 52.96 | 52.94 | 52.99 |
| 66.05 | 66.02 | 66.08 |
| 51.04 | 51.02 | 51.07 |
| 69.07 | 69.05 | 69.11 |
| 29.04 | 29.02 | 29.05 |

37. A method according to claim 35, wherein the group comprises each of the ranges of the table.

38. A method according to claim 35, wherein the group consists in the ranges of the table.

39. A method according to claim 24, wherein the transfer line is connected to an end of an endotracheal tube installed on the person.

40. A method according to claim 24, further comprising heating the transfer line.
